(19) United States
(12) Patent Application Publication
(10) Pub. No.: US 2025/0134994 A1
(43) Pub. Date: May 1, 2025

(54) IMMUNOGENIC MRNA DELIVERY VEHICLES

(71) Applicant: Corner Therapeutics, Inc., Watertown, MA (US)

(72) Inventors: Andrew N. CORNFORTH, Carlisle, MA (US); Emily A. GOSSELIN, West Roxbury, MA (US)

(73) Assignee: Corner Therapeutics, Inc., Watertown, MA (US)

(21) Appl. No.: 18/836,248

(22) PCT Filed: Feb. 6, 2023
(86) PCT No.: PCT/US2023/062063

§ 371 Date: Aug. 6, 2024

Related U.S. Application Data
(60) Provisional application No. 63/307,578, filed on Feb. 7, 2022.

(51) Int. Cl. A61K 39/39; A61K 9/1272; A61K 9/51; A61K 31/4745; A61K 47/26; A61K 47/54

(52) U.S. Cl. CPC A61K 39/39; A61K 9/1272; A61K 9/5123; A61K 31/4745; A61K 47/26; A61K 47/543

(57) ABSTRACT

The present disclosure relates to lipid-based delivery vehicles for mRNA vaccines, which include a lysophosphatidylcholine (LPC) compound for enhancing vaccine immunogenicity. The present disclosure also relates to methods for use of the mRNA vaccines.

### CROSS-REFERENCE TO RELATED APPLICATIONS

This application claims priority to and benefit of U.S. Provisional Patent Application No. 63/307,578, filed Feb. 7, 2022, which is incorporated herein by reference in its entirety.

### FIELD

The present disclosure relates to lipid-based delivery vehicles for mRNA vaccines, which include a lysophosphatidylcholine (LPC) compound for enhancing vaccine immunogenicity. The present disclosure also relates to methods for use of mRNA vaccines.

### BACKGROUND

mRNA vaccines have several advantages over traditional vaccines. For instance, mRNA vaccines can be quickly developed, cheaply produced, and safely administered (Pardi et al., Nat Reg Drug Discov, 17(4):261-279, 2018). However, naked mRNA is unstable and is quickly degraded after administration. Fortunately, advances in mRNA chemistry and delivery systems have enabled the rapid production of several effective mRNA COVID-19 vaccines (Hou et al., Nature Review Materials, 6:1078-1094, 2021).

Even so, there is room for improvement of lipid-based formulations for delivery of mRNA vaccines. Specifically, more immunogenic and/or less reactogenic formulations are desirable.

### BRIEF SUMMARY

The present disclosure relates to lipid-based delivery vehicles for mRNA vaccines, which include a lysophosphatidylcholine (LPC) compound for enhancing vaccine immunogenicity. The present disclosure also relates to methods for use of mRNA vaccines.

### BRIEF DESCRIPTION OF THE DRAWINGS

FIG. 1A-B show that lipid nanoparticles (LNPs) loaded with both 22:0 Lyso PC and mRNA remain nanoparticle sized (<125 nm) and are relatively uniform in diameter (PDI<0.3). FIG. 1A shows that LNPs loaded with mRNA are larger than LNPs lacking mRNA, and that adding 22:0 LPC to LNPs increases the size of mRNA loaded LNPs. FIG. 1B shows that all formulations tested contain LNPs with relatively uniform sizes.

FIG. 2A-2F show that loading LNPs with 22:0 Lyso PC increases immunogenicity of LNP formulations by allowing for hyperactivation of human monocyte-derived dendritic cells (moDCs). Viability of human moDCs cultured with LNPs loaded with 50 μM or 100 μM 22:0 Lyso PC relative to human moDCs treated with R848 alone (exemplary PAMP) is shown in FIG. 2A and FIG. 2B, respectively. IL-1β secretion by human moDCs cultured with LNPs loaded with 50 μM or 100 μM 22:0 Lyso PC, with or without the addition of R848 is shown in FIG. 2C and FIG. 2D, respectively. IL-6 secretion by human moDCs cultured with LNPs loaded with 50 μM or 100 μM 22:0 Lyso PC, with or without the addition of R848 is shown in FIG. 2E and FIG. 2F, respectively. LNPs were prepared with mRNA encoding GFP or without mRNA, and were prepared with various levels of 22:0 Lyso PC (0%, 30%, 40% molar ratios of 22:0 Lyso PC in LNPs). LNPs without 22:0 Lyso PC (LNP 0) were dosed to provide similar total lipid levels as LNPs loaded with 22:0 Lyso PC. mRNA dose was similar across formulation types (about 5 g/mL).

FIG. 3A-3D show that loading 22:0 Lyso PC into LNPs containing mRNA does not prevent mRNA translation. Expression of green fluorescent protein (GFP) was assessed as a percentage of GFP-positive moDCs after culture for 48 hrs in the presence of LNPs loaded with 50 μM or 100 μM 22:0 Lyso PC, as shown in FIG. 3A and FIG. 3B, respectively. Expression of GFP was also assessed as median fluorescence intensity (MFI) in moDCs after culture for 48 hrs in the presence of LNPs loaded with 50 μM or 100 μM 22:0 Lyso PC, as shown in FIG. 3C and FIG. 3D, respectively. LNPs were prepared with mRNA encoding GFP or without mRNA, and LNPs were prepared with various levels of 22:0 Lyso PC (0%, 30%, 40% molar ratios of 22:0 Lyso PC in LNPs). LNPs without 22:0 Lyso PC (LNP 0) were dosed to provide similar total lipid levels as LNPs loaded with 22:0 Lyso PC. mRNA dose was similar across formulation types (about 5 g/mL).

FIG. 4A-4H show that loading 22:0 Lyso PC into LNPs containing mRNA increases activation marker and antigen presenting molecule expression by moDCs. Specifically, loading 22:0 Lyso PC into LNPs increases CD86 expression (FIG. 4A-4B), CD40 expression (FIG. 4C-4D), HLA-DR expression (FIG. 4E-4F), and HLA-ABC expression (FIG. 4G-4H) in moDCs treated with 50 μM or 100 μM 22:0 Lyso PC. LNPs were prepared with various levels of 22:0 Lyso PC (0%, 30%, 40% molar ratios of 22:0 Lyso PC in LNPs). LNPs without 22:0 Lyso PC (LNP 0) were dosed to provide similar total lipid levels as LNPs loaded with 22:0 Lyso PC. mRNA dose was similar across formulation types (about 5 g/mL).

FIG. 5A-5B show that LNPs loaded with 22:0 Lyso PC and OVA mRNA promote activation of naïve transgenic murine T cells (OT-I T cells) expressing T cell receptors that recognize an OVA epitope. FIG. 5A shows IL-1β secretion by murine bone marrow-derived dendritic cells (BMDCs) cultured with LNPs loaded with 50 μM or 100 μM 22:0 Lyso PC and 0, low, or high amounts of OVA mRNA, and in the presence or absence of R848. FIG. 5B shows IFNγ secretion by OVA-specific, OT-I T cells co-cultured for 72 hrs with BMDCs that had been hyperactivated for 48 hrs with LNPs containing OVA mRNA and loaded with 50 μM 22:0 Lyso PC, with or without the addition of R848. LNPs were prepared with various levels of 22:0 Lyso PC (0%, 40% molar ratios of 22:0 Lyso PC in LNPs). LNPs without 22:0 Lyso PC (LNP 0) were dosed to provide similar total lipid levels as LNPs loaded with 22:0 Lyso PC. OVA mRNA doses tested included: 0, low dose (about 0.25 g/mL), and high dose (about 2.5 g/mL).

FIG. 6A-B show that LNPs loaded with 22:0 Lyso PC and OVA mRNA reactivate OVA-specific T cells from mice that had been previously immunized against OVA. FIG. 6A shows IFNγ secretion by T cells previously exposed to OVA and co-cultured for 96 hrs with BMDCs that had been hyperactivated for 24 hrs with LNPs loaded with 50 μM 22:0 Lyso PC, with or without the addition of R848. FIG. 6B shows IFNγ secretion by T cells previously exposed to OVA and co-cultured for 96 hrs with BMDCs that had been hyperactivated for 48 hrs with LNPs loaded with 50 μM 22:0 Lyso PC, with or without the addition of R848. LNPs were prepared with various levels of 22:0 Lyso PC (0%, 40% molar ratios of 22:0 Lyso PC in LNPs). LNPs without 22:0 Lyso PC (LNP 0) were dosed to provide similar total lipid levels as LNPs loaded with 22:0 Lyso PC. OVA mRNA doses tested included: 0, low dose (about 0.25 g/mL), and high dose (about 2.5 g/mL).

### DETAILED DESCRIPTION

The present disclosure relates to lipid-based delivery vehicles for mRNA vaccines, which include a lysophosphatidylcholine (LPC) compound for enhancing vaccine immunogenicity. The present disclosure also relates to methods for use of the mRNA vaccines.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., a molecular weight of about 900 daltons, refers to a molecular weight of from 810 daltons to 990 daltons).

An “effective amount” or a “sufficient amount” of a substance is that amount sufficient to effect beneficial or desired results, including clinical results, and, as such, an “effective amount” depends upon the context in which it is being applied. For instance, in the context of administering an immunogenic composition, an effective amount contains sufficient antigen, and one or both of a lysophosphatidylcholine (LPC) compound and a PRR agonist, to stimulate an immune response against the antigen (e.g., antigen-reactive antibody and/or cellular immune response).

The terms “individual” and “subject” refer to mammals. “Mammals” include, but are not limited to, humans, non-human primates (e.g., monkeys), farm animals, sport animals, rodents (e.g., mice and rats), and pets (e.g., dogs and cats). In some embodiments, the subject is a human patient, such as a human patient suffering from cancer and/or an infectious disease.

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). As an example, when used in reference to an LPC, an isolated LPC is at least 90%, 95%, 96%, 97%, 98% or 99% pure as determined by thin layer chromatography, or gas chromatography. As a further example, when used in reference to a recombinant protein, an isolated protein refers to a protein that has been removed from the culture medium of the host cell that produced the protein.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions are intended to be sterile.

“Excipients” as used herein include pharmaceutically acceptable excipients, carriers, vehicles or stabilizers that are nontoxic to the cell or mammal being exposed thereto at the dosages and concentrations employed. Often the physiologically acceptable excipient is an aqueous pH buffered solution.

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. Antigens when present in the compositions of the present disclosure can be synthetic or isolated from nature. Antigens suitable for administration in the methods of the present disclosure include any molecule capable of eliciting an antigen-specific B cell or T cell response. Haptens are included within the scope of “antigen.” A “hapten” is a low molecular weight compound that is not immunogenic by itself but is rendered immunogenic when conjugated with a generally larger immunogenic molecule (carrier).

“Polypeptide antigens” can include purified native peptides, synthetic peptides, recombinant peptides, crude peptide extracts, or peptides in a partially purified or unpurified active state (such as peptides that are part of attenuated or inactivated viruses, microorganisms or cells), or fragments of such peptides. Polypeptide antigens are preferably at least eight amino acid residues in length.

The term “agonist” is used in the broadest sense and includes any molecule that activates signaling through a receptor. In some embodiments, the agonist binds to the receptor. For instance, a TLR8 agonist binds to a TLR8 receptor and activates a TLR8-signaling pathway.

“Alkyl” refers to monovalent saturated aliphatic hydrocarbyl groups. Cx alkyl refers to an alkyl group having x number of carbon atoms. Cx-Cy alkyl or Cx-y alkyl refers to an alkyl group having between x number and y number of carbon atoms, inclusive.

“Alkylene” refers to divalent saturated aliphatic hydrocarbyl groups.

“Alkenyl” refers to monovalent hydrocarbyl groups having at least one double bond (>C═C<). Cx alkenyl refers to an alkenyl group having x number of carbon atoms. Cx-Cy alkenyl or Cx-y alkenyl refers to an alkenyl group having between x number and y number of carbon atoms, inclusive.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 2-fold to 2,000-fold, or from 5-fold to 500-fold or over, or from 2, 5, 10, 50, or 100-fold to 500, 1,000, 2,000, 5,000, or 10,000-fold.

Conversely, “inhibition” of a response or parameter includes reducing and/or repressing that response or parameter when compared to otherwise same conditions except for a parameter of interest, or alternatively, as compared to another condition (e.g., decrease in abnormal cell proliferation after administration of a composition comprising a LPC compound and one or more of a pathogen recognition receptor agonist, an antigen, and human dendritic cells, as compared to the administration of a placebo composition or no treatment). For example, “inhibition” of an immune response means a decrease in the response. Depending upon the parameter measured, the decrease may be from 2-fold to 2,000-fold, or from 5-fold to 500-fold or over, or from 2, 5, 10, 50, or 100-fold to 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher level of DC hyperactivation” refers to a level of DC hyperactivation as a consequence of a treatment condition (comprising a LPC compound of the present disclosure) that is at least 2, 3, 4, 5, 6, 7, 8, 9, or 10-fold above a level of DC hyperactivation as a consequence of a control condition (e.g., no LPC, PGPC, oxPAPC, etc.). Likewise, a “lower level of DC hyperactivation” refers to a level of DC hyperactivation as a consequence of a treatment condition (comprising a LPC compound of the present disclosure) that is at least 2, 3, 4, 5, 6, 7, 8, 9, or 10-fold below a level of DC hyperactivation as a consequence of a control condition (e.g., no LPC, PGPC, oxPAPC, etc.).

As used herein the term “immunization” refers to a process that increases a mammalian subject's reaction to antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen, enhances or potentiates an immune response to the antigen in the mammalian recipient upon exposure.

The terms “treating” or “treatment” of a disease refer to executing a protocol, which may include administering one or more therapeutic agents to an individual (human or otherwise), in an effort to obtain beneficial or desired results in the individual, including clinical results. Beneficial or desired clinical results include, but are not limited to, alleviation or amelioration of one or more signs or symptoms of a disease, diminishment of extent of disease, stabilized (i.e., not worsening) state of disease, preventing spread of disease, delay or slowing of disease progression, amelioration or palliation of the disease state, and remission (whether partial or total). “Treatment” also can mean prolonging survival as compared to expected survival of an individual not receiving treatment. Further, “treating” and “treatment” may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure, and specifically includes protocols that have only a palliative effect on the individual. “Palliating” a disease or disorder means that the extent and/or undesirable clinical manifestations of the disease or disorder are lessened and/or time course of progression of the disease or disorder is slowed, as compared to the expected untreated outcome.

### I. Lysophosphatidylcholine Compounds

A “lysophosphatidylcholine” (LPC) or “lysophosphatidylcholine molecule” refers to a glycerol molecule bearing one phosphocholine group on a hydroxyl group of the glycerol and bearing one acyl group on one of the other two hydroxyl groups of the glycerol. The remaining hydroxyl group is unsubstituted.

In some embodiments, the isolated lysophosphatidylcholine (LPC) with a single acyl chain is of the form:

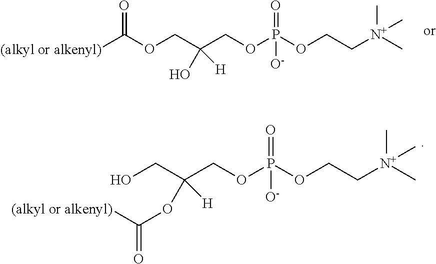

In some embodiments, the isolated lysophosphatidylcholine (LPC) with a single acyl chain is of the form:

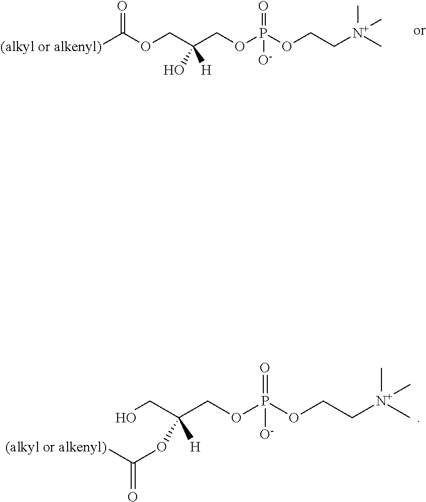

The alkyl or alkenyl chain, together with the carbonyl carbon, forms an acyl chain which is one carbon atom longer than the alkyl or alkenyl chain. For example, a (C23 alkyl)-C(═O)— group forms a C24 acyl chain. Thus, when the group “(alkyl or alkylene)” is a C12-C23 alkyl group (such as a C12-C19 alkyl group or a C20-C23 alkyl group), the (C12-C23 alkyl-C(═O)— group forms a C13-C24 acyl chain (such as a C13-C20 acyl chain or a C21-C24 acyl chain). When the group “(alkyl or alkylene)” is a C12-C23 alkenyl group (such as a C12-C19 alkenyl group or a C20-C23 alkenyl group), the (C12-C23 alkenyl-C(═O)— group forms a C13-C24 acyl chain (such as a C13-C20 acyl chain or a C21-C24 acyl chain). Acyl chains can be referred to as saturated acyl or unsaturated acyl to distinguish between alkyl-containing and alkenyl-containing acyl groups. Standard delta notation or omega notation can be used to indicate the position of one or more double bonds in an unsaturated acyl chain.

Lysophosphatidylcholine (LPC) compounds of the present disclosure have a single acyl chain in which the acyl chain is a C13-C22 acyl chain or a C13-C24 acyl chain. In some embodiments, the acyl chain is a C18-C22 acyl chain or a C21-C24 acyl chain. In some preferred embodiments, the acyl chain is a C22 acyl chain. Names and structures of exemplary LPC compounds for inclusion in LNPs of the present disclosure, as well as their Chemical Abstract Service (CAS) Registry Numbers are listed as Compounds #30-#43, optionally #30-#42 of Table I of International Application No. PCT/US2022/071664, which is incorporated herein by reference. Several methods are known for synthesis of lysophospholipids (see, e.g., D'Arrigo et al, “Synthesis of lysophospholipids,” Molecules, 15(3):1354-77, 2010; and Yang et al., “Lysophosphatidylcholine synthesis by lipase-catalyzed ethanolysis,” J Oleo Sci., 64(4):443-7, 2015, and the references cited therein). Additionally, many lysophospholipids are commercially available.

### II. Pathogen Recognition Receptor Agonists

Compositions and methods of the present disclosure may further comprise a pathogen recognition receptor (PRR) agonist. In some embodiments, the PRR agonist comprises an agonist of a toll-like receptor (TLR), a NOD-like receptor (NLR), a RIG-I-like receptor (RLR), or a C-type lectin receptor (CLR). In other embodiments, the PRR agonist comprises a cytosolic DNA sensor (CDS) or a stimulator of IFN genes (STING). In some embodiments, the PRR agonist comprises a TLR7/8 agonist.

### A. TLR7/8 Agonists

The term “TLR7/8 agonist” as used herein refers to an agonist of TLR7 and/or TLR8. In one aspect, the TLR7/8 agonist is a TLR7 agonist. In another aspect, the TLR7/8 agonist is a TLR8 agonist. In a further aspect, the TLR7/8 agonist is an agonist of both TLR7 and TLR8. TLR7/8 agonists of the present disclosure are suitable for hyperactivating human dendritic cells in the presence of LPC.

In some aspects, the TLR7/8 agonist is a small molecule. In some embodiments, the TLR7/8 agonist is a small molecule with a molecule weight of 900 daltons or less, or a salt thereof. That is, the small molecule TLR7/8 agonist is not a large molecule like a recombinant protein or a synthetic oligonucleotide, which is regulatable by the U.S. FDA's Center for Biologics Evaluation and Research. Rather the small molecule TLR7/8 agonist is regulatable by the FDA's Center for Drug Evaluation and Research. In some embodiments, the small molecule has a molecule weight of from about 90 to about 900 daltons. In some embodiments, the TLR7/8 agonist comprises an imidazoquinoline compound. In some preferred embodiments, the TLR7/8 agonist comprises resiquimod (R848).

### B. Other PRR Agonists

In some aspects, the pathogen recognition receptor (PRR) agonist comprises a toll-like receptor (TLR) agonist with the proviso that the TLR agonist does not comprise a TLR7/8 agonist. In some embodiments, the TLR agonist comprises an agonist of one or more of TLR2, TLR3, TLR4, TLR5, TLR9 and TLR13. In some embodiments, the PRR agonist is a TLR2/6 agonist, such as Pam2CSK4. In other embodiments, the TLR agonist is a TLR4 agonist such as monophosphoryl lipid A (MPLA). However, in preferred embodiments, the TLR agonist is not an agonist of TLR2, TLR4 and/or TLR9. For instance, in preferred embodiments, the TLR9 agonist is not a TLR4 ligand such as LPS (endotoxin).

In other aspects, the PRR agonist comprises a NOD-like receptor (NLR) agonist. In further aspects, the PRR agonist comprises a RIG-I-like receptor (RLR) agonist. In additional aspects, the PRR agonist comprises a C-type lectin receptor (CLR) agonist. In still further aspects, the PRR agonist comprises a CDS agonist or a STING agonist.

III. mRNA Encoding an Antigen

Compositions and methods of the present disclosure comprise an mRNA encoding an antigen or are otherwise suitable for use with a formulation comprising an mRNA encoding an antigen. In some embodiments, the antigen is a proteinaceous antigen. The terms “polypeptide” and “protein” are used interchangeably herein in reference to antigens that comprise peptide chains that are at least 8 amino acids in length. In some embodiments, the antigen is from 8 to 1800 amino acids, 9 to 1000 amino acids, or 10 to 100 amino acids in length. The polypeptide may be post-translationally modified such as by phosphorylation, hydroxylation, sulfonation, palmitoylation, and/or glycosylation.

In some embodiments, the antigen is a tumor antigen that comprises the amino acid sequence of at least one full length protein or fragment thereof. In some embodiments, the tumor antigen comprises an amino acid sequence or fragment thereof from an oncoprotein. In some embodiments, the mammalian antigen is a neoantigen or encoded by a gene comprising a mutation relative to the gene present in normal cells from a mammalian subject. Neoantigens are thought to be particularly useful in enabling T cells to distinguish between cancer cells and non-cancer cells (see, e.g., Schumacher and Schreiber, Science, 348:69-74, 2015). In other embodiments, the tumor antigen comprises a viral antigen, such as an antigen of a cancer-causing virus.

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the antigen is a microbial antigen. In some embodiments, the microbial antigen comprises a viral antigen, a bacterial antigen, a protozoan antigen, a fungal antigen, or combinations thereof. In some embodiments, the microbial antigen comprises a surface protein or other antigenic subunit of a microbe.

In some preferred embodiments, the mRNA comprises a 5′ untranslated region (5′UTR) at the 5′ end of the coding region and a 3′ untranslated region (3′UTR) at the 3′ end of the coding region. In some preferred embodiments, the mRNA comprises one or both of a 5′ cap structure and a polyA tail.

### IV. Lipid-Based Delivery Vehicles

Compositions and methods of the present disclosure comprise a lipid-based delivery vehicle for an mRNA vaccine. In some embodiments, the vehicle is a lipid nanoparticle (LNP). In other embodiments, the vehicle is a lipid that forms a complex with the mRNA (RNA-Lipoplex).

In some embodiments, the LNP comprises a first phospholipid (lysophosphatidylcholine with a single C13-C24 acyl chain [LPC:C13-C24] and at least one lipid selected from the group consisting of an ionizable lipid, a cationic lipid, a second phospholipid, a pegylated lipid, a structural lipid, and mixtures thereof. In some embodiments, the at least one lipid comprises an ionizable lipid. In some embodiments, the at least one lipid comprises a cationic lipid. In some embodiments, the at least one lipid comprises a second phospholipid. In some embodiments, the at least one lipid comprises a pegylated lipid. In some embodiments, the at least one lipid comprises a structural lipid. In some embodiments, the at least one lipid comprise an ionizable lipid, a second phospholipid, a pegylated lipid, and a structural lipid.

In some embodiments, the lipid component of RNA-Lipoplex comprises one or more lipids. In some preferred embodiments, the one or more lipids comprise a first lipid and a second lipid, wherein the first lipid is distinct from the second lipid. In some embodiments, the first lipid is a cationic lipid and the second lipid is a neutral or anionic lipid.

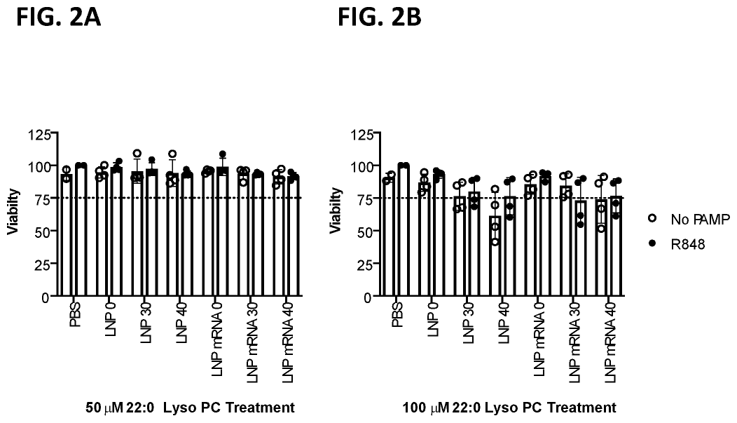

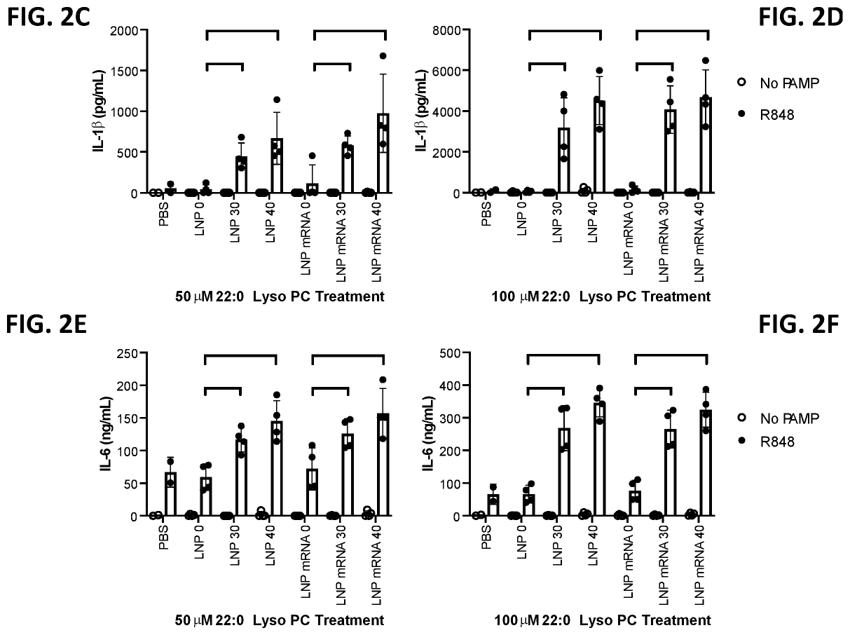

Structure of lipids suitable for use in the lipid-based mRNA delivery vehicles of the present disclosure are shown below (reproduced from FIG. 2 of Hou et al., Nature Review Materials, 6:1078-1094, 2021).

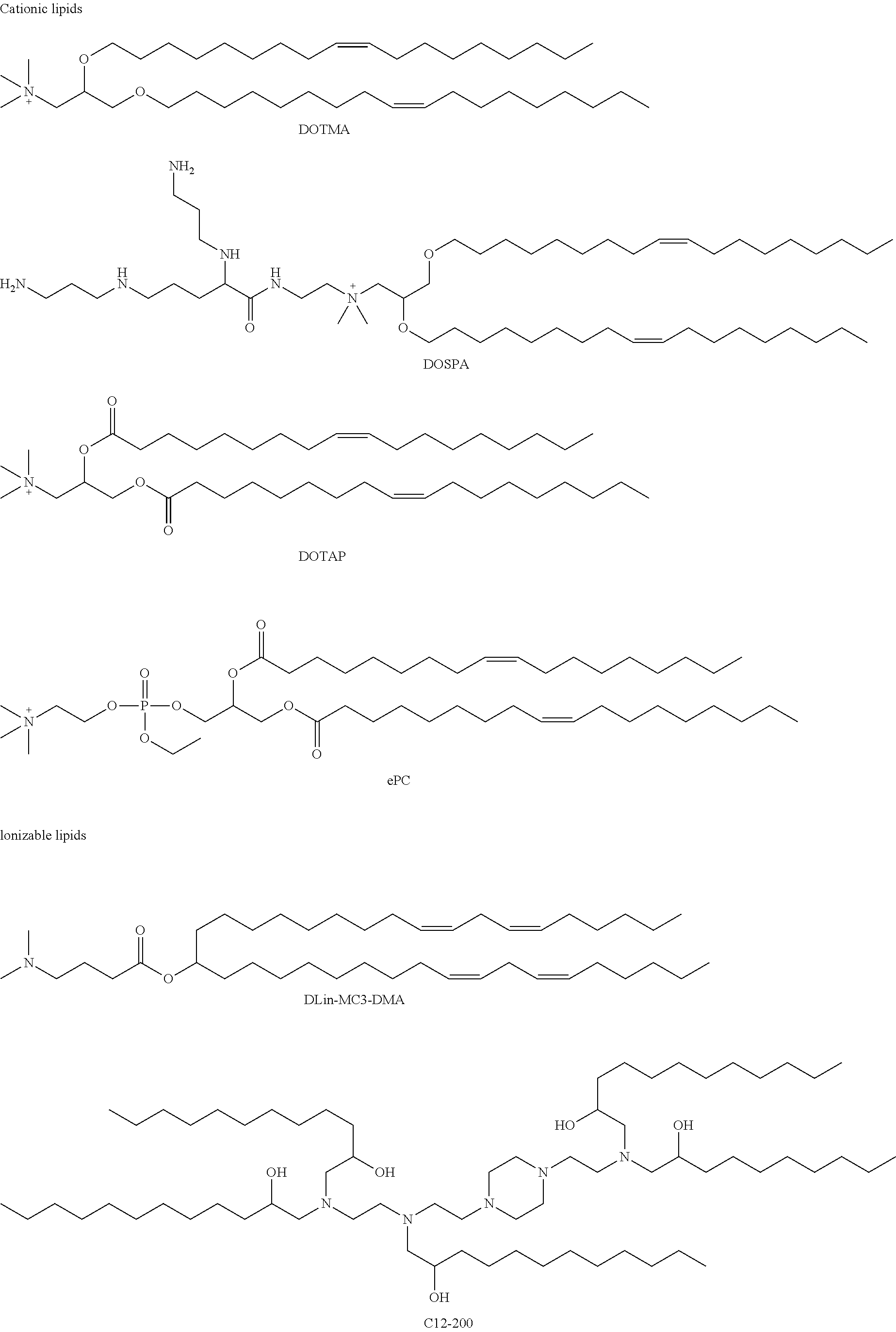

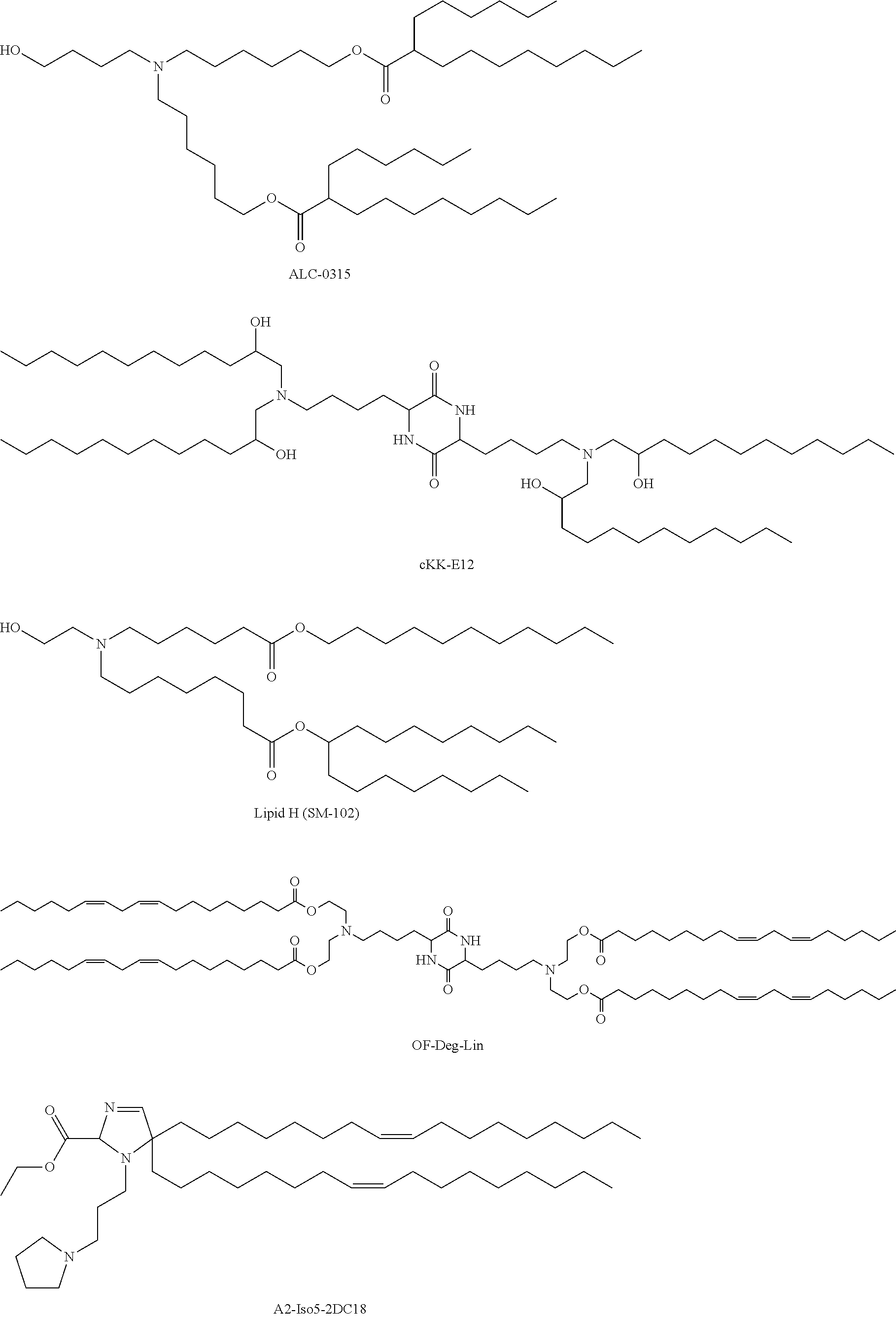

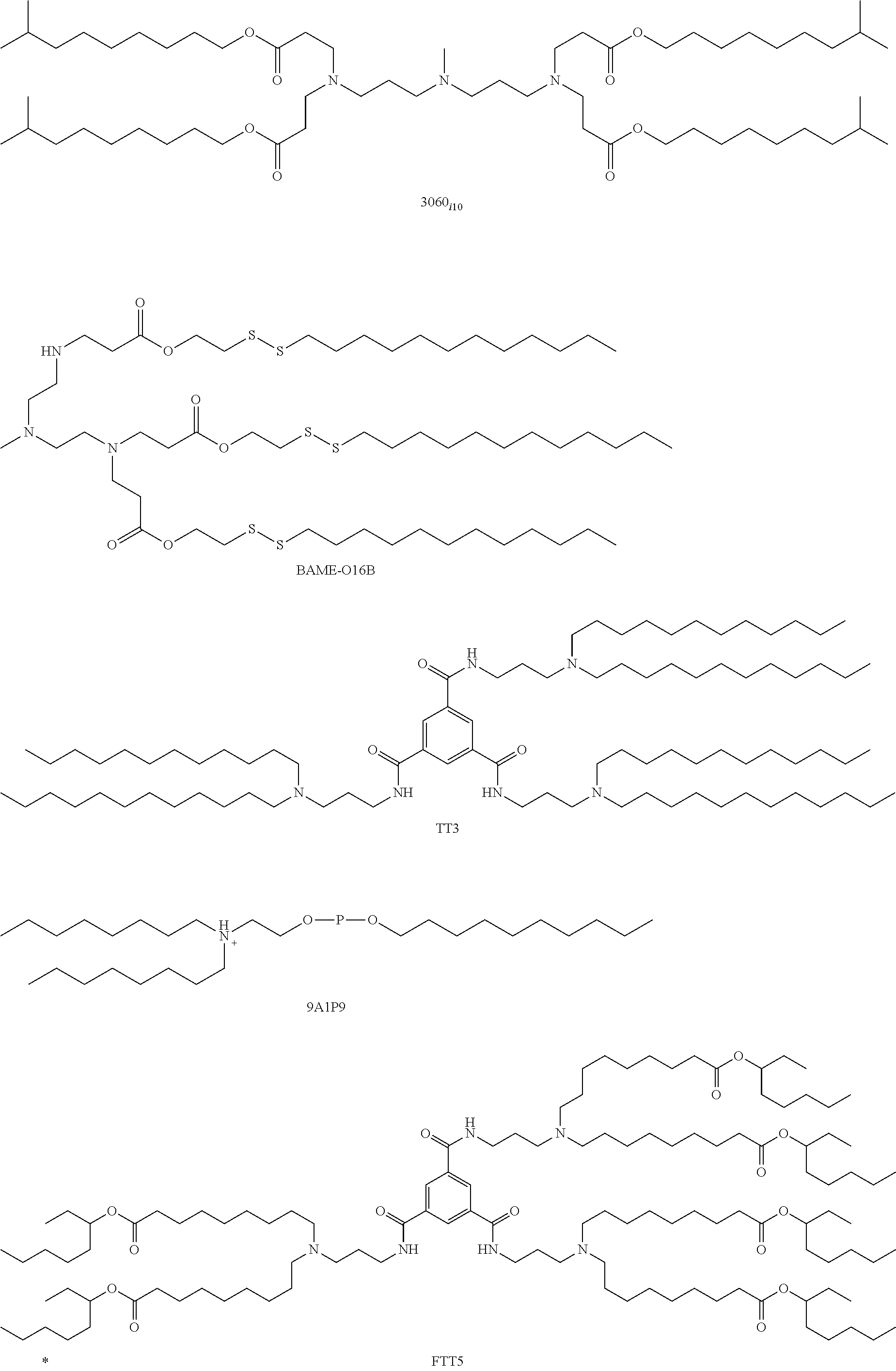

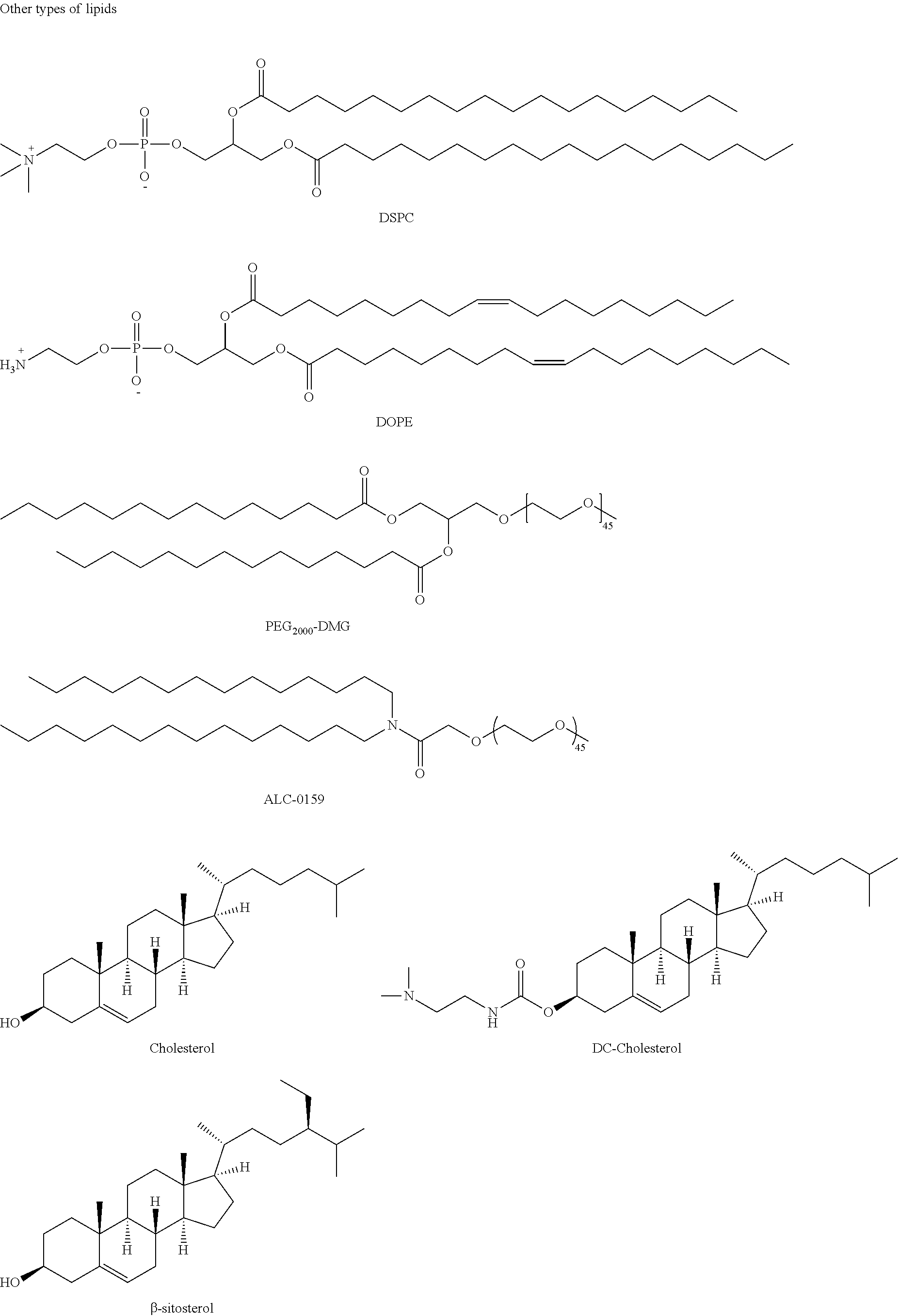

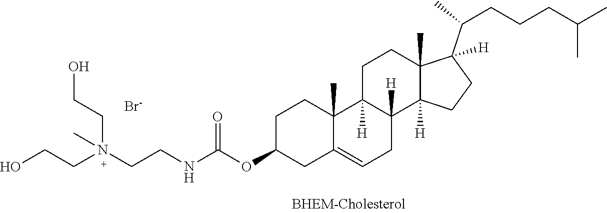

### V. Pharmaceutical Formulations

Some compositions of the present disclosure are pharmaceutical formulations comprising a pharmaceutically acceptable excipient. Pharmaceutical formulations of the present disclosure may be in the form of a solution or a suspension. Alternatively, the pharmaceutical formulations may be a dehydrated solid (e.g., freeze dried or spray dried solid). The pharmaceutical formulations of the present disclosure are preferably sterile, and preferably essentially endotoxin-free. The term “pharmaceutical formulations” is used interchangeably herein with the terms “medicinal product” and “medicament”. In some embodiments, the pharmaceutical formation comprises specific ratios of the various components based on the intended purpose of the formulation.

Pharmaceutically acceptable excipients of the present disclosure include for instance, solvents, buffering agents, tonicity adjusting agents, bulking agents, and preservatives (See, e.g., Pramanick et al., Pharma Times, 45:65-77, 2013). In some embodiments, the pharmaceutical formulations may comprise an excipient that functions as one or more of a solvent, a buffering agent, a tonicity adjusting agent, and a bulking agent (e.g., sodium chloride in saline may serve as both an aqueous vehicle and a tonicity adjusting agent).

In some embodiments, the pharmaceutical formulations comprise an aqueous vehicle as a solvent. Suitable vehicles include for instance sterile water, saline solution, phosphate buffered saline, and Ringer's solution. In some embodiments, the composition is isotonic.

The pharmaceutical formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical formulations may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical formulations may comprise a preservative. Suitable preservatives include for instance antioxidants and antimicrobial agents. However, in preferred embodiments, the pharmaceutical formulation is prepared under sterile conditions and is in a single use container, and thus does not necessitate inclusion of a preservative.

The pharmaceutical formulations of the present disclosure are suitable for parenteral administration. That is the pharmaceutical formulations of the present disclosure are not intended for enteral administration (e.g., not by orally, gastrically, or rectally).

### VI. Methods of Use

In some aspects, the present disclosure relates to methods of use of any one of the compositions or formulations described herein. The methods of use are suitable for a plurality of uses involving stimulating an immune response. In some embodiments, the methods of use comprise methods of treating cancer. In some embodiments, the methods of use comprise methods of inhibiting abnormal cell proliferation. In some embodiments, the methods of use comprise methods of treating or preventing an infectious disease. The methods comprise administering an effective amount of a formulation or a composition described herein to an individual in need thereof to achieve a specific outcome. The individual is a mammalian subject, such as a human patient. In other embodiments, the individual a non-human patient. In some embodiments, the individual is a canine patient. That is in some embodiments, the methods of use involve clinical uses, while in other embodiments the methods of use involve pre-clinical and/or veterinary uses. For preclinical uses, the mammalian subject may be a non-human primate (e.g., monkey or ape) or a rodent (e.g., mouse or rat). For veterinary uses the mammalian subject may be a farm animal (e.g., cow), a sport animal (e.g., horse), a or a pet (e.g., companion animal such as a dog or cat).

### A. Stimulation of an Immune Response

In brief, the present disclosure provides methods of stimulating an immune response in an individual, comprising administering to the individual a composition or formulation described herein in an amount sufficient to stimulate an immune response in the individual. “Stimulating” an immune response (used interchangeably with “eliciting” and immune response), means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response comprises one or more of the group consisting of: stimulating cytokine production; stimulating B lymphocyte proliferation; stimulating interferon pathway-associated gene expression; stimulating chemoattractant-associated gene expression; and stimulating dendritic cell DC maturation. Methods for measuring stimulation of an immune response are known in the art.

For instance, the present disclosure provides methods of inducing an antigen-specific immune response in an individual by administering to the individual a composition or formulation described herein in an amount sufficient to induce an antigen-specific immune response in the individual. In preferred embodiments, the composition or formulation comprises the antigen. In some embodiments, the composition or formulation is administered to a tissue of the individual comprising the antigen. The immune response may comprise one or both of an antigen-specific antibody response and an antigen-specific cytotoxic T lymphocyte (CTL) response. “Inducing” an antigen-specific antibody response means increasing titer of the antigen-specific antibodies above a threshold level such as a pre-administration baseline titer or a seroprotective level. “Inducing” an antigen-specific CTL response means increasing frequency of antigen-specific CTL found in peripheral blood above a pre-administration baseline frequency.

Analysis (both qualitative and quantitative) of the immune response can be by any method known in the art, including, but not limited to, measuring antigen-specific antibody production (including measuring specific antibody subclasses), activation of specific populations of lymphocytes such as B cells and helper T cells, production of cytokines such as IFN-alpha, IFN-gamma, IL-6, IL-12 and/or release of histamine. Methods for measuring antigen-specific antibody responses include enzyme-linked immunosorbent assay (ELISA). Activation of specific populations of lymphocytes can be measured by proliferation assays, and with fluorescence-activated cell sorting (FACS). Production of cytokines can also be measured by ELISA. In some embodiments, methods of stimulating an immune response comprise stimulation of interleukin-1beta (IL-1β) secretion, interferon-gamma (IFN-γ) secretion, and/or tumor necrosis factor-alpha (TNF-α) secretion by monocyte-derived dendritic cells or peripheral blood mononuclear cells. In some preferred embodiments, at least 50%, 55%, 60%, 65%, 70% or 75% of the cells contacted with a composition of the present disclosure remain viable at 40-56 hours (or about 48 hours) post-contact. In some preferred embodiments, at least 75% of the cells contacted with a composition of the present disclosure remain viable at 40-56 hours (or about 48 hours) post-contact.

In some embodiments, the methods are suitable for stimulating an anti-tumor immune response. In other embodiments, the methods are suitable for stimulating an anti-microbe immune response. In some embodiments, the anti-microbe response is an anti-bacterial immune response. In some embodiments, the anti-microbe response is an anti-fungal immune response. In some embodiments, the anti-microbe response is, an anti-viral immune response. In some embodiments, the anti-microbe response is an anti-protozoan immune response.

### B. Treating or Preventing Disease

The present disclosure further provides methods of treating or preventing a disease in an individual, comprising administering to the individual a composition or formulation described herein in an amount sufficient to treat or prevent a disease in the individual. In some embodiments, the disease is cancer. In some embodiments, the disease is abnormal cell proliferation. In other embodiments, the disease is an infectious disease. In one aspect, the methods may comprise administering a composition to a subject in need thereof.

In some embodiments, the methods involve treating cancer in an individual or otherwise treating a mammalian subject with cancer. In some embodiments, the cancer is a hematologic cancer, such as a lymphoma, a leukemia, or a myeloma. In other embodiments, the cancer is a non-hematologic cancer, such as a sarcoma, a carcinoma, or a melanoma. In some embodiments, the cancer is malignant.

In some embodiments, the methods involve inhibiting abnormal cell proliferation in an individual. “Abnormal cell proliferation” refers to proliferation of a benign tumor or a malignant tumor. The malignant tumor may be a metastatic tumor.

In some embodiments, the methods involve treating or preventing an infectious disease in an individual. In some embodiments, the infectious disease is caused by a viral infection. In other embodiments, the infectious disease is caused by a bacterial infection. In further embodiments, the infectious disease is caused by a fungal infection. In still further embodiments, the infectious disease is caused by a protozoal infection. Of particular importance are infectious diseases caused by zoonotic pathogens that infect humans as well as other animals such as mammals or birds. In some embodiments, the zoonotic pathogen is transmitted to humans via an intermediate species (vector).

### Enumerated Embodiments

1. A composition comprising an mRNA encapsulated in a lipid nanoparticle (LNP), wherein the mRNA comprises a coding region of an antigen, and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

2. A composition comprising an mRNA and a TLR7/8 agonist encapsulated in a lipid nanoparticle (LNP), wherein the mRNA comprises a coding region of an antigen, and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

3. A composition comprising an mRNA encapsulated in a lipid nanoparticle (LNP), and a TLR7/8 agonist, wherein the mRNA comprises a coding region of an antigen, and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

4. A composition comprising a TLR7/8 agonist encapsulated in a lipid nanoparticle (LNP), and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

5. A composition comprising a lipid nanoparticle (LNP) and a TLR7/8 agonist, wherein the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

6. The composition of any one of embodiments 1-5, wherein the at least one lipid comprises an ionizable lipid, a second phospholipid, a pegylated lipid, and a structural lipid.

7. The composition of any one of embodiments 1-6, wherein the ionizable lipid comprises:
i) SM-102 or analogs or derivatives thereof; and/or
ii) ALC-0315 or analogs or derivatives thereof.

8. The composition of any one of embodiments 1-7, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.

9. The composition of any one of embodiments 1-7, wherein the pegylated lipid comprises polyethylene glycol [PEG]2000 dimyristoyl glycerol [DMG].

10. The composition of any one of embodiments 1-9, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.

11. The composition of any one of embodiments 1-9, wherein the structural lipid comprises cholesterol.

12. The composition of any one of embodiments 1-11, wherein the second phospholipid comprises:
i) a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin; and
ii) one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

13. The composition of any one of embodiments 1-11, wherein the second phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
combinations thereof.

14. The composition of any one of embodiments 1-13, wherein the second phospholipid comprises 1,2-distearoyl-sn-glycero-3-phosphocholine (DSPC).

15. The composition of any one of embodiments 1-14, wherein the composition further comprises at least one excipient.

16. The composition of embodiment 15, wherein the excipient comprises sucrose.

17. A composition comprising:
i) an mRNA complexed with one or more lipids (RNA-Lipoplex); and
ii) a lysophosphatidylcholine (LPC) with a single C13-C24 acyl chain, wherein the mRNA comprises a coding region of an antigen, and the one or more lipids comprise a first lipid and a second lipid.

18. A composition comprising:
i) an mRNA complexed with one or more lipids (RNA-Lipoplex);
ii) a lysophosphatidylcholine (LPC) with a single C13-C24 acyl chain; and
iii) a TLR7/8 agonist,
   wherein the mRNA comprises a coding region of an antigen, and the one or more lipids comprise a first lipid and a second lipid.

19. The composition of embodiment 17 or embodiment 18, wherein the first lipid is a cationic lipid, and the second lipid is a neutral or anionic lipid.

20. The composition of embodiment 19, wherein the cationic lipid comprises one or both of:
i) 1,2-di-O-octadecenyl-3-trimethylammonium propane (DOTMA) or analogs or derivatives thereof; and
ii) 1,2-dioleoyl-3-trimethylammonium propane (DOTAP) or analogs or derivatives thereof.

21. The composition of embodiment 19 or embodiment 20, wherein the neutral or anionic lipid comprises:
i) 1,2-di-(9Z-octadecenoyl)-sn-glycero-3-phosphoethanolamine (DOPE) or analogs or derivatives thereof; and/or
ii) cholesterol or analogs or derivatives thereof; and/or
iii) 1,2-dioleoyl-sn-glycero-3-phosphocholine (DOPC) or analogs or derivatives thereof.

22. The composition of any one of embodiments 1-21, wherein the acyl chain of the LPC is a C21-C24 acyl chain.

23. The composition of any one of embodiments 1-21, wherein the acyl chain of the LPC is a C22 acyl chain.

24. The composition of any one of embodiments 1-23, wherein the acyl chain of the LPC is fully saturated.

25. The composition of embodiment 24, wherein the LPC comprises 1-behenoyl-2-hydroxy-sn-glycero-3-phosphocholine [LPC(22:0)].

26. The composition of any one of embodiments 1-25, wherein the TLR7/8 agonist is a small molecule with a molecule weight of 900 daltons or less

27. The composition of any one of embodiments 1-25, wherein the TLR7/8 agonist comprises an imidazoquinoline compound.

28. The composition of embodiment 27, wherein the TLR7/8 agonist comprises resiquimod (R848).

29. The composition of any one of embodiments 1-28, wherein the LPC comprises LPC(22:0), and the TLR7/8 agonist comprises resiquimod (R848).

30. The composition of any one of embodiments 1-28, wherein the antigen is a tumor antigen.

31. The composition of any one of embodiments 1-28, wherein the tumor antigen is a neoantigen.

32. The composition of any one of embodiments 1-28, wherein the antigen comprises a microbial antigen.

33. The composition of embodiment 32, wherein the microbial antigen comprises a viral antigen, a bacterial antigen, a protozoan antigen, or a fungal antigen.

34. The composition of embodiment 32, wherein the microbial antigen comprises a surface antigen.

35. The composition of any one of embodiments 1-34, wherein the mRNA comprises a 5′ untranslated region (5′UTR) at the 5′ end of the coding region and a 3′ untranslated region (3′UTR) at the 3′ end of the coding region.

36. The composition of any one of embodiments 1-35, wherein the mRNA comprises a 5′ cap structure.

37. The composition of any one of embodiments 1-36, wherein the mRNA comprises a polyA tail.

38. The composition of any one of embodiments 1-37, wherein the composition does not comprise lipopolysaccharide (LPS) or monophosphoryl lipid A (MPLA).

39. The composition of any one of embodiments 1-38, wherein the composition does not comprise oxidized 1-palmitoyl-2-arachidonoyl-sn-glycero-3-phosphorylcholine (oxPAPC) or a species of oxPAPC.

40. The composition of embodiment 39, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl]2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl]2-(trimethylazaniumyl)ethyl phosphate (POVPC), [(2R)-2-(4-carboxybutanoyloxy)-3-hexadecanoyloxy propyl]2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl]2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl]2-(trimethylazaniumyl)ethyl phosphate (PEIPC) and/or 1-palmitoyl-2-azelaoyl-sn-glycero-3-phosphocholine (PAzePC).

41. The composition of any one of embodiments 1-40, wherein the composition does not comprise an antigen.

42. A pharmaceutical formulation comprising the composition of any one of embodiments 1-41, and a pharmaceutically acceptable excipient.

43. A method for production of hyperactivated dendritic cells, the method comprising contacting the dendritic cells with an effective amount of the composition of any of the preceding embodiments to produce hyperactivated dendritic cells, wherein the hyperactivated dendritic cells secrete IL-1beta without undergoing cell death within about 48 hours of exposure.

44. The method of embodiment 43, wherein the dendritic cells are contacted in vivo with the composition.

45. The method of embodiment 43, wherein the dendritic cells are contacted ex vivo with the composition.

46. A pharmaceutical formulation comprising at least 10\hat{ }3, 10\hat{ }4, 10\hat{ }5 or 10\hat{ }6 of the hyperactivated dendritic cells produced by the method of embodiment 45, and a pharmaceutically acceptable excipient.

47. A method of stimulating an immune response against an antigen, comprising administering an effective amount of the pharmaceutical formulation of embodiment 42 or embodiment 46 to an individual in need thereof to stimulate the immune response against the antigen.

48. A method of treating cancer, comprising administering an effective amount of the pharmaceutical formulation of embodiment 42 or embodiment 46 to an individual in need thereof to treat the cancer.

49. A method of inhibiting abnormal cell proliferation, comprising administering an effective amount of the pharmaceutical formulation of embodiment 42 or embodiment 46 to an individual in need thereof to inhibit abnormal cell proliferation.

50. A method of treating or preventing an infectious disease, comprising administering an effective amount of the pharmaceutical formulation of embodiment 42 to an individual in need thereof to treat or prevent the infectious disease.

51. The method of embodiment 50, wherein the infectious disease is a viral disease.

52. The method of embodiment 51, wherein the infectious disease is a bacterial disease.

53. The method or pharmaceutical formulation of any one of embodiments 43-49, wherein the dendritic cells are mammalian cells.

54. The method or pharmaceutical formulation of embodiment 53, wherein the mammalian cells are human cells.

55. The method of any one of embodiments 47-53, wherein the individual is mammal.

56. The method of embodiment 55, wherein the mammal is a human.

57. The method of embodiment 55, wherein the mammal is a dog or a cat.

58. The composition, formulation, or method or use of any one of embodiments 1-57, wherein the composition does not comprise a protein.

59. The composition, formulation, method or use of any one of embodiments 1-158, wherein the LNP has an effective diameter of less than about 500 nanometers, optionally from about 5 to about 500 nanometers, optionally from about 10 to about 400 nanometers, optionally from about 20 to about 300 nanometers, or optionally from about 25 to about 250 nanometers.

60. The composition, formulation, method or use of embodiment 59, wherein the LNP has an effective diameter of less than about 250 nanometers.

61. The composition, formulation, method or use of embodiment 60, wherein the LNP has an effective diameter of less than about 125 nanometers.

62. The composition, formulation, method or use of embodiment 61, wherein the LNP has an effective diameter of from about 20 to about 120 nanometers.

### EXAMPLES

Abbreviations: BMDC (bone marrow-derived dendritic cell); CDS (cytosolic DNA sensor); CLR (C-type lectin receptor); DAMP (damage-associated molecular pattern); DC (dendritic cell); DMG-PEG2000 (1,2-dimyristoyl-rac-glycero-3-methoxypolyethylene glycol-2000); DSPC (1,2-distearoyl-sn-glycero-3-phosphocholine); GFP (green fluorescent protein); HOdiA-PC (1-Palmitoyl-2-(5-hydroxy-8-oxo-6-octenedioyl)-sn-glycero-3-phosphatidylcholine); HOOA-PC (1-palmitoyl-2-(5-hydroxy-8-oxooct-6-enoyl)-sn-glycero-3-phosphocholine); IFNγ (interferon-gamma); IL-1b/IL1-beta/IL-1β (Interleukin-1beta); KOdiA-PC (1-(Palmitoyl)-2-(5-keto-6-octene-dioyl)phosphatidylcholine); KOOA-PC (1-palmitoyl-(5-keto-8-oxo-6-octenoyl)-sn-glycero-3-phosphocholine); LNP (lipid nanoparticle); LPC/Lyso PC (lysophosphatidylcholine); Lyso PC(22:0) (1-behenoyl-2-hydroxy-sn-glycero-3-phosphocholine); LPS (lipopolysaccharide); moDC (monocyte-derived dendritic cell); MPLA (monophosphoryl lipid A); NLR (NOD-like receptor); OVA (ovalbumin); oxPAPC (oxidized 1-palmitoyl-2-arachidonyl-sn-glycero-3-phosphorylcholine); PAMP (pathogen-associated molecular pattern); PBMCs (peripheral blood mononuclear cells); PDI (polydispersity index); PGPC (1-palmitoyl-2-glutaryl-sn-glycero-3-phosphocholine); POVPC (1-palmitoyl-2-(5′-oxo-valeroyl)-sn-glycero-3-phosphocholine); PRR (pathogen recognition receptor); RLR (RIG-I-like receptor); R848 (resiquimod); SM102 ((8-[(2-hydroxyethyl)[6-oxo-6-(undecyloxy)hexyl]amino]-octanoic acid), 1-octylnonyl ester); STING (stimulator of IFN genes); TNFα (tumor necrosis factor-alpha); and TLR (toll-like receptor).

Although the present disclosure has been described in some detail by way of illustration and example for purposes of clarity and understanding, it will be apparent to those skilled in the art that certain changes and modifications may be practiced. Therefore, the examples should not be construed as limiting the scope of the present disclosure, which is delineated by the appended claims.

### Example 1: Combination of a Lysophosphatidylcholine (LPC) with a Single Acyl Chain and a TLR7/8 Agonist Hyperactivates Mammalian Peripheral Blood Mononuclear Cells

This example describes the hyperactivation of canine and human peripheral blood mononuclear cells (PBMCs) with a lipid DAMP in combination with a small molecule PAMP.

### Materials and Methods

Isolation of PBMCs from Human and Canine Whole Blood. PBMCs were isolated from whole blood using density gradient centrifugation with Ficoll-Paque PLUS (Cytivia). Whole blood was diluted 1:1 with PBS, layered on top of Ficoll-Paque PLUS and centrifuged at 1000×g for 30 minutes at room temperature. PBMCs were collected, washed twice in PBS, and incubated with Ack lysis buffer (Lonza) to remove any remaining red blood cells.

Cell Culture and Stimulation. Immediately following isolation, PBMCs were plated in RPMI medium containing 10% FBS, 50 units/mL penicillin, 50 mg/mL streptomycin, 2 mM L-glutamine, 1 mM sodium pyruvate, and 50 mM beta-mercaptoethanol (R10 media). Cells were plated at 1×10⁵ (canine cells) or 1×10⁶ (human cells) per well in 96-well flat bottom tissue culture plates. Lyophilized Vaccigrade R848 (Invivogen) was reconstituted and diluted according to manufacturer's recommendations and added to cells at a final concentration of 1 μg/mL. Immediately following, 22:0 LYSO PC (1-behenoyl-2-hydroxy-sn-glycero-3-phosphocholine) was added to cells at a final concentration of 82.5 μM. Additional innate agonists were diluted in R10 media according to manufacturer's recommendations and added to the cells as follows: human GM-CSF (Peprotech) was added at a final concentration of 10 ng/mL; 2′3′ cGAMP (Invivogen) was added at a final concentration of 15 μg/mL; LPS, serotype O55:B5 (Enzo Life Sciences) was added at a final concentration of 1 μg/mL; Alum hydroxide (Invivogen) was added at a final concentration of 30 μg/mL. Cells were incubated at 37° C., 5% C02 for two days. Cell cultures were then used for endpoint analyses.

Endpoint Analyses. After culturing PBMCs with PAMPs and DAMPs for two days, supernatant and cell samples were collected for analysis. Cells in culture were pelleted by centrifugation at 400×g for 5 minutes. Half of the media volume in the wells was collected for cytokine quantification by Enzyme-Linked Immunosorbent Assay (ELISA) or Lumit™ Bioluminescent assay, while the remaining media and cells were used to quantify cell viability by assessing metabolic activity.

Quantification of Cytokine Secretion. IL-1β secretion from human PBMCs was assessed using one of the following kits: ELISA MAX Deluxe Set Human IL-1β kit (Biolegend), Invitrogen Human IL-1β kit, or the Lumit™ Human IL-1β Immunoassay (Promega). IFNγ secretion from human PBMCs was assessed using the ELISA MAX Deluxe Set Human IFNγ (Biolegend) and TNFα secretion from human PBMCs was assessed using the Human TNFα Uncoated ELISA kit (Invitrogen). ELISAs were performed according to manufacturer's instructions with the following modifications: i) total sample+buffer volume for incubation was reduced from 100 μL to 50 μL; ii) the top standard was prepared at 500 μg/mL, with two-fold dilutions to 7.8 μg/mL; and iii) sample incubation was completed overnight at 4 C on an orbital shaker. Lumit™ assays were performed according to manufacturer's instructions. IL-1β secretion from canine PBMCs was assessed using the Canine IL-1β/IL-1F2 DuoSet ELISA (R&D) according to manufacturer's instructions with the following modifications: i) total sample+buffer volume for incubation was reduced from 100 μL to 50 μL; ii) sample incubation was completed overnight at 4° C. on an orbital shaker. For all ELISAs, absorbance was measured at 450 nm, with a 570 nm correction, using a Spectramax M5e plate reader (Molecular Devices). For Lumit™ assays, luminescence was measured on all wavelengths using a Spectramax M5e plate reader (Molecular Devices) with an integration time of 500 ms. To determine cytokine concentrations in supernatants, sample concentrations were interpolated using a standard curve via 4PL analysis on GraphPad Prism 9 (GraphPad Software). The interpolated results of samples were then adjusted for any dilutions made to the supernatant.

Quantification of Cell Viability. Cell viability was assessed by quantifying the presence of ATP as an indicator of metabolically active cells using the CellTiter-Glo Luminescent Cell Viability Assay (Promega). Metabolic activity was assessed following manufacturer's instructions. The CellTiter-Glo reagent was mixed with the cell pellets and fresh media then transferred to a white, opaque 96-well plate. Luminescence was measured on all wavelengths on a Spectramax M5e plate reader (Molecular Devices) using an integration time of 500 ms. Percent viability was calculated relative to the control condition of PBMCs treated with R848.

Statistical Analyses. For each condition, cells from each donor were plated for testing in triplicate. For cytokine quantification, triplicate values were used for interpolation and data was plotted as total concentration (pg/mL) or fold change per donor relative to the control condition of R848 alone. For viability quantification, each donor triplicate was averaged, and the average was used as one donor measurement. Multiple donors were tested and each data point on the column graphs represents the value for a donor. To test for differences in test conditions, test results were compared to the control condition of R848 alone. P-values were calculated using a mixed-effects one-way ANOVA, with corrections for multiple comparisons using a Dunnett's test.

Results—Treatment with 22:0 LYSO PC and R848 Hyperactivates Canine PBMCs

The combination of 22:0 LYSO PC (DAMP) and the TLR7/8 agonist R848 (PAMP) was previously found to have potent hyperstimulatory activity in human moDCs In order to assess whether this hyperstimulatory activity translates to other clinically relevant species, the ability of 22:0 LYSO PC+R848 to hyperactivate PBMCs isolated from canine whole blood was assessed. For each data set, PBMCs from multiple donors were used in lieu of moDCs due to the lack of canine-specific reagents available to induce bone fide canine moDCs. In brief, PBMCs were isolated from whole blood using density gradient centrifugation and then cultured for two days with the hyperactivating stimuli of interest.

After two days in culture, hyperactivation was assessed by quantification of IL-1β in cell culture supernatants and measurement of cell viability. When treated with 22:0 LYSO PC and R848 together, canine PBMCs secreted comparable or higher levels of IL-1β compared to every other stimuli tested, both as concentration per mL, as well as fold change per donor relative to R848 alone. Consistent with previous studies showing that monocytes, which make up 5-10% of PBMCs, can release IL-1β in response to activation with R848, canine PBMCs had elevated levels of IL-1β secretion with R848 alone compared to untreated cells. The pyroptotic combination of LPS+Alum elicited high levels of IL-1β as expected. Notably, while PGPC+R848 elicited similar levels of IL-1β compared to R848 alone, neither GM-CSF nor 2′3′cGAMP induced significant IL-1β secretion from canine PBMCs compared to untreated cells.

Although IL-1β can be detected one day after hyperactivation of canine PBMCs in cell culture supernatants, cell viability was evaluated two days post-hyperactivation to ensure enduring viability after IL-1β secretion. 22:0 LYSO+R848 did not significantly reduce relative cell viability. Interestingly, PGPC in combination with R848 proved to be somewhat toxic to canine PBMCs, although it was not observed to be toxic to human moDCs or human PBMCs. However, it is important to note that interpreting observations made viability concerns from testing mixed cell populations iscan be challenging, as the viability of a specific cell populations of interest (in this case, monocytes that we suspect are responding to IL-1□) cannot be determined from the mixture. Together these data demonstrate that 22:0 LYSO+R848 elicits high levels of IL-1β secretion from canine PBMCs, which is indicative of hyperactivation.

Results—Treatment with 22:0 LYSO PC and R848 Hyperactivates Human PBMCs

Hyperactivation experiments were also performed with PBMCs isolated from whole blood obtained from human donors. In brief, PBMCs were isolated from whole blood by density gradient centrifugation from multiple human donors and cultured for two days with the hyperactivating stimuli of interest.

Human PBMCs, like human moDCs and canine PBMCs, secreted IL-1l at levels higher or comparable to all other stimuli tested. Similar to canine PBMCs, human PBMCs secreted IL-1β in response to R848 alone due to monocyte activation, and this was elevated by addition of 22:0 LYSO PC. The pyroptotic combination of LPS+Alum elicited high levels of IL-1β as expected. Consistent with observations in canine PBMCs, PGPC+R848 did not induce substantially higher levels of IL-1β than R848 alone. GM-CSF did not induce levels of IL-1β secretion from human PBMCs significantly above background levels produced by untreated cells.

Viability of human PBMCs was also assessed two days post-hyperactivation to ensure enduring viability of human PBMCs after IL-1β secretion. No significant decreases in human PBMC viability were observed after treatment with any of the stimuli. However, interpreting observations made from testing mixed cell populations is challenging, as the viability of a specific cell population of interest (in this case, monocytes) cannot be determined from results obtained with the mixture. Together these data demonstrate that both human and canine PBMCs are hyperactivated by 22:0 LYSO PC+R848. Interestingly, canine PBMCs are hyperactivated to a greater extent by 22:0 LYSO PC+R848 than by PGPC+R848.

Because activated human PBMCs can secrete other cytokines in addition to IL-1β, the secretion of the pro-inflammatory cytokines IFNγ and TNFα in cell culture supernatants was measured two days post-hyperactivation. The combination of 22:0 LYSO PC+R848 induced the highest fold change per donor in both IFNγ secretion and TNFα secretion relative to R848 alone as compared to all other stimuli tested. Notably, although LPS+Alum induced high levels of IL-1β secretion from human PBMCs, this combination of stimuli did not induce a fold increase in IFNγ or TNFα secretion. Moreover, neither GM-CSF nor 2′3′cGAMP elicited a substantial fold change in IFNγ secretion over R848 alone. These data indicate that the combination of 22:0 LYSO PC+R848 is superior at inducing secretion of the proinflammatory cytokines IFNγ and TNFα from human PBMCs.

### Example 2: Incorporation of a LPC with a Single Acyl Chain into Lipid Nanoparticles in the Presence of mRNA for Hyperactivation of Mammalian Dendritic Cells

This example describes the preparation and testing of lipid nanoparticles (LNPs) containing a lysophosphatidylcholine (LPC) compound with a single acyl chain (e.g., 22:0 Lyso PC) and mRNA encoding an antigen. The LPC/mRNA-loaded LNPs are suitable for hyperactivating mammalian dendritic cells in combination with a small molecule PAMP (e.g., R848).

### Materials and Methods

LNP Synthesis. LNPs were prepared by combining the following components with or without 1-behenoyl-2-hydroxy-sn-glycero-3-phosphocholine (CAS Registry No. 125146-65-8, referred to herein as “22:0 Lyso PC”) (Avanti):
(8-[(2-hydroxyethyl)[6-oxo-6-(undecyloxy)hexyl]amino]-octanoic acid), 1-octylnonyl ester (CAS Registry No. 2089251-47-6, referred to herein as “SM102”) (Cayman Chemical);
1,2-distearoyl-sn-glycero-3-phosphocholine (CAS Registry No. 816-94-4, referred to herein as “DSPC”) (Avanti);
cholesterol (Sigma); and
1,2-dimyristoyl-rac-glycero-3-methoxypolyethylene glycol-2000 (CAS Registry No. 160743-62-4, referred to herein as “DMG-PEG2000”) (Avanti).
   Table 2-1 details the molar percentages of each component for each LNP formulation tested. LNPs were either prepared without 22:0 Lyso PC, or loaded with 20%, 30%, or 40% molar ratios of 22:0 Lyso PC in LNPs to determine if the 22:0 Lyso PC loading could be intentionally varied. LNPs were made either without mRNA, with mRNA encoding green fluorescent protein (GFP), or with mRNA encoding ovalbumin (OVA) at varying loading levels to determine if including 22:0 Lyso PC would impact LNP mRNA loading.

LNPs were synthesized using the NanoAssemblr Ignite instrument (Precision Nanosystems). Lipids were first dissolved in ethanol and then combined following the molarity compositions shown in Table 2-1. Lipids in ethanol were combined with sodium citrate buffer (pH 4) containing mRNA at a 1:3 volumetric ratio, at a flow rate of 12 mL/min. Enhanced GFP mRNA (Trilink) was added to a concentration of 0.053 mg/mL in sodium citrate buffer for loading into LNPs. OVA mRNA (Trilink) was added to a concentration of 0.035 mg/mL or 0.004 mg/mL in sodium citrate buffer for loading LNPs. LNPs were washed in 10 volumes of PBS, pH 7.4 to remove residual ethanol, and then concentrated using Amicon 10K MWCO centrifugal filters.

TABLE 2-1
Percent Molarity of Lipids in LNP Formulations

| | LNP | 22:0 | 22:0 | 22:0 |
|---|---|---|---|---|
| | Vehicle | Lyso PC | Lyso PC | Lyso PC |
| Lipid | LNP 0 | LNP 20 | LNP 30 | LNP 40 |
| SM102 | 35% | 35% | 35% | 30% |
| DSPC | 33% | 13% | 3% | 0% |
| Cholesterol | 30.5% | 30.5% | 30.5% | 28.5% |
| DMG-PEG2000 | 1.5% | 1.5% | 1.5% | 1.5% |
| 22:0 Lyso PC | 0% | 20% | 30% | 40% |

LNP Characterization. Loading of 22:0 Lyso PC into LNPs was assessed using HPLC. LNPs in PBS were frozen at −20° C. until quantification. LNPs were dissolved by adding 1 part ethanol to the LNPs in PBS. A seven point standard curve of 22:0 Lyso PC was prepared in 1:1 ethanol:PBS added to match sample preparation. Standards and samples were filtered through a 0.45 μm filter prior to running on the HPLC. HPLC quantification was performed on using an Agilent 1260 Infinity II HPLC equipped with a 1260 Infinity II Evaporative Light Scattering Detector. A Luna 5 μm NH2 100 Å, 150×4.6 mm LC Column (Phenomenex) with a column temperature of 30° C. was used to detect samples. Two eluents were used: A, 100% water; and B, 100% acetonitrile. An initial mobile phase composed of 5%/95% A/B was used to load the column, with a gradient reaching 24%/76% A/B after 2.5 min. A more shallow gradient was used from 2.5 to 6 minutes, with A/B slowly reaching 25%/75% during that time frame. A post time of 3 min was used to return the gradient to starting conditions prior to the next sample run. The flow rate was set to 1 mL/min, and the injection volume was 2.5 μL for samples and standards. The evaporative light scattering detector (ELSD) used an evaporator temperature of 50° C., a nebulizer temperature of 30° C., and a gas flow rate of 0.9 standard L/min. Agilent CDS 2.6 software was used for HPLC instrument control, data acquisition, and processing.

Loading of mRNA into LNPs was quantified using a RiboGreen assay (ThermoFisher) following the manufacturer's protocol. Samples were diluted to fall within the range of the standard curve. LNPs were lysed using Triton X-100 to assess encapsulation of mRNA into LNPs. Both total mRNA and encapsulated mRNA were quantified. The size of the LNPs was assessed using dynamic light scattering (DLS) on the NanoBrook Omni (Brookhaven). LNPs were diluted 1:10 in PBS before running on the DLS. Three 90 second measurements were recorded for each sample

Human monocyte-derived dendritic cell (moDC) generation. Human monocytes were isolated from Leukopaks purchased from Miltenyi Inc. (San Jose, CA) using the StraightFrom® Leukopak® CD14 microbead kit according to the manufacturer's instructions. Monocytes were then aliquoted and frozen in FBS containing 10% dimethyl sulfoxide. Ahead of testing, monocytes were thawed and cultured in RPMI medium containing 10% FBS, 50 units/mL penicillin, 50 mg/mL streptomycin, 2 mM L-glutamine, 1 mM sodium pyruvate, 50 mM beta-mercaptoethanol, 10 mM HEPES, and Gibco MEM non-essential amino acids (R10 media). To differentiate monocytes into moDCs, recombinant human GM-CSF (50 ng/mL) and IL-4 (25 ng/mL) were added to R10 media. Cells were cultured for 6 days with GM-CSF and IL-4, with an additional feeding with R10 media containing GM-CSF and IL-4 on day 3.

Human monocyte-derived dendritic cell (moDC) hyperactivation. Six days after differentiation, moDCs were collected and counted. Cells were plated into 96-well flat-bottom plates at 1×10⁵ cells/well. Cells were treated with or without 1 g/mL R848 (final), with or without LNPs loaded with a hyperactivating lipid (or vehicle control) in the presence or absence of eGFP mRNA. Hyperactivation induced by LNPs was measured after 48 hrs in culture with the LNPs. Cell Viability was assessed using the LDH CyQuant Kit (Invitrogen) following manufacturer's instructions. The IL-1β and IL-6 Lumit assays (Promega) were used to measure IL-1β and IL-6 present in moDC cell culture supernatants. Experimental conditions were tested in triplicate and the mean result from two human donors was plotted. Data represent results from two experiments.

In addition to hyperactivation, GFP expression in the moDCs was quantified using flow cytometry on a BD FACS Symphony A3 device. moDCs were collected after 48 hrs in culture with LNPs, and stained with Live/Dead stain to identify live cells, followed by staining with antibodies specific for CD11c, CD40, CD86, HLA-DR, and HLA-ABC. Live cells were selected for analysis, and then CD11c⁺ cells were assessed for GFP expression to determine if the mRNA of the LNPs could be translated into GFP protein when LNPs were loaded with 22:0 Lyso PC. Antigen presentation was assessed using antibodies specific for HLA-DR and HLA-ABC to determine if hyperactivation would interfere with antigen presentation. Activation was assessed by staining for CD40 and CD86 to determine if hyperactivation would increase expression of activation markers.

Generation of murine bone marrow-derived FLT3L-DCs (BMDCs). Leg femur and tibia were removed from mice, cut with scissors, and flushed into sterile tubes. Bone marrow suspension was treated with ACK Lysis Buffer for 1 min, then passed through a 40 μm cell strainer. Cells were counted and resuspended in media consisting of complete IMDM containing 10% FBS, penicillin and streptomycin, and supplements of L-glutamine and sodium pyruvate (I10). Cells were then plated at 8×10⁶ cells/well in a P12 plate. Recombinant mouse FLT3L (Miltenyi) was added to cultures at 200 ng/mL. Differentiated cells were used for subsequent assays on day 8. The efficiency of differentiation was monitored by flow cytometry using a BD Symphony A3 device, and CD11c⁺MHC-II⁺ cells were routinely above 80% of living cells. For each experiment, 5 to 15 mice were used to generate BMDCs.

Hyperactivation of murine bone marrow-derived FLT3L-DCs and T cell co-culture. BMDCs were harvested on day 8 and day 9 post differentiation, washed with PBS and re-plated in complete IMDM media (I10) at a concentration of 2×10⁵ cells/well. Cells were cultured in the presence or absence of LNPs loaded with 22:0 Lyso PC LNPs at 50 μM, and with or without OVA mRNA at either a high or low dose of mRNA (Table 2-2). At 24 and 48 hrs post stimulation, supernatants and BMDCs were collected. IL-1β cytokine secretion by BMDCs was measured using sandwich ELISAs (Invitrogen) following manufacturer's instructions. About 2×10⁴ hyperactivated BMDCs were seeded in wells of a round bottom 96 well-plate for co-culture with T cells.

CD8+ T cells were collected from the spleens and lymph nodes of transgenic OT-I mice, which express T cell receptors (TCRs) specific for an ovalbumin peptide (residues 257-264) presented by H2-K^{b}. All CD8 T cells of OT-I mice are naïve cells specific to the ovalbumin peptide. The T-cell co-culture was set-up by adding 6×10⁴ CD8+ T-cells to each well containing BMDCs. Hyperactivated BMDCs were co-cultured with T cells for 72 hrs, after which the co-culture supernatants were collected.

Additionally, CD8+ T cells were collected from the spleens and lymph nodes of mice previously immunized with ovalbumin (OVA) in incomplete freund's adjuvant (IFA) using a mouse CD8 T cell isolation kit (Miltenyi) following the manufacturer's protocol. Immunization elicits a CD8+ T cell population containing OVA-specific T cells that were previously exposed to antigen at physiologically relevant levels. The T-cell co-culture was set-up by adding 1.6×10⁵ CD8+ T-cells to each well containing BMDCs. Hyperactivated BMDCs and CD8+ T cells were co-cultured for 96 hrs, after which time co-culture supernatants were collected.

IFNγ secretion by CD8+ T cells in response to OVA presented by BMDCs was assessed using the Mouse Lumit IFNγ kit (Promega) according to manufacturer's instructions. Luminescence was measured across all wavelengths for 500 ms, using a Spectramax M5e plate reader (Molecular Devices). To determine IFNγ concentrations in supernatants, sample concentrations were interpolated using a standard curve via 4PL analysis on GraphPad Prism 9 (GraphPad Software). The interpolated results of samples were then adjusted for any dilutions made to the supernatant.

### Results

Lipid nanoparticles (LNPs) have become an important vaccine delivery tool, especially in the context of mRNA vaccines, which have played a large role in the fight against COVID-19 worldwide. LNPs are particularly useful for delivering mRNA cargo into cells. However, while LNP-based mRNA vaccines are effective at inducing antibody responses to the antigen(s) they encode, mRNA vaccines often elicit limited antigen-specific T cell responses, which negatively impacts their efficacy and longevity. In order to address this problem, the present disclosure describes the addition of a lysophosphatidylcholine (LPC) compound with a single acyl chain, such as 22:0 Lyso PC, to an LNP formulation containing an mRNA encoding an antigen to enhance its immunogenicity.

Based on the structure of the exemplary lipid, 22:0 Lyso PC was contemplated to be incorporable into LNPs and that modulation of various LNP components would affect the physical characteristics, as well as, the biological activity of the LNPs loaded with 22:0 Lyso PC. Both loading level and the number of loaded LNPs were contemplated to be key variables affecting the 22:0 Lyso PC payload delivered to cells.

22:0 Lyso PC can be loaded into LNPs. LNPs were prepared with or without 22:0 Lyso PC by combining the following: SM102, DPSC, cholesterol, and DMG-PEG2000. The input molar ratios for these LNP formulations are listed in Table 2-1. As 22:0 Lyso PC is most structurally similar to DSPC, 22:0 Lyso PC replaced DSPC in the formulation as the amount of 22:0 Lyso PC added to the LNPs increased. To determine if the loading level of 22:0 Lyso PC could be intentionally varied, and to understand how the loading levels would impact the biological activity of the LNPs, several different LNP compositions containing varying levels of 22:0 Lyso PC were prepared. LNPs were prepared either without 22:0 Lyso PC (LNP 0), or loaded with 20% (LNP 20), 30% (LNP 30), or 40% (LNP 40) molar ratios of 22:0 Lyso PC. LNPs were also made either without mRNA, or with mRNA encoding GFP (GFP mRNA), or with mRNA encoding OVA (OVA mRNA) at varying loading levels to determine if including 22:0 Lyso PC would impact mRNA loading into LNPs.

Table 2-2 details the loading ratios of 22:0 Lyso PC to mRNA for the formulations tested. Various loading levels of mRNA/22:0 Lyso PC were tested to identify conditions in which both the mRNA and 22:0 Lyso PC would be biologically active. In all cases, mRNA and 22:0 Lyso PC were both be loaded into LNPs, as assessed by Ribogreen and HPLC, respectively. With the exception of 22:0 Lyso PC LNP 20, mRNA loading for all LNP formulations was >75%, and 22:0 Lyso PC loading was >80%.

TABLE 2-2
Relative Loading Levels of LNP Formulations\hat{ }

| | Cargo Input | mRNA | 22:0 Lyso |
|---|---|---|---|
| | Ratio: mRNA/ | loading | PC loading |
| LNP Formulation | 22:0 Lyso PC | efficiency | efficiency |
| 22:0 Lyso PC LNP | — | 75.5% | — |
| 0:GFP mRNA | | | |
| 22:0 Lyso PC LNP | 0.110 | 69.4% | 76.7% |
| 20:GFP mRNA | | | |
| 22:0 Lyso PC LNP | 0.074 | 76.6% | 82.1% |
| 30:GFP mRNA | | | |
| 22:0 Lyso PC LNP | 0.055 | 77.7% | 92.4% |
| 40:GFP mRNA | | | |
| 22:0 Lyso PC LNP | 0.044 | 103.0% | 86.5% |
| 40:OVA mRNA High Dose | | | |
| 22:0 Lyso PC LNP | 0.005 | 115.7% | 86.3% |
| 40:OVA mRNA Low Dose | | | |

\hat{ }LNPs were prepared that did not contain mRNA (LNP 0, LNP 30, LNP 40). These LNPs loaded 22:0 Lyso PC at similar efficiencies to the LNPs containing mRNA.

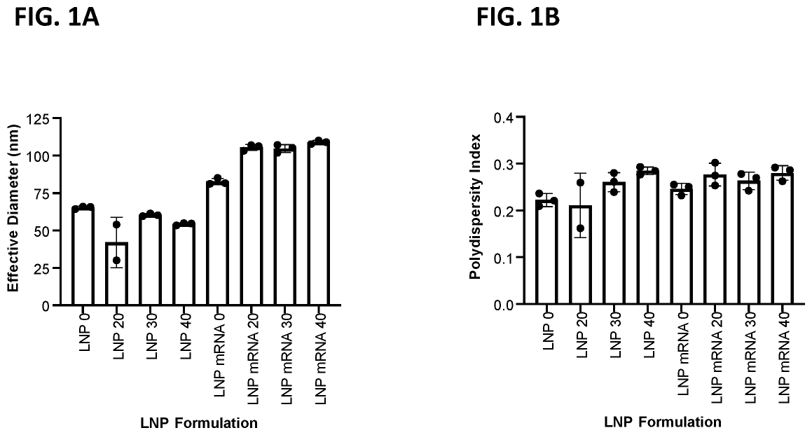

Importantly, both 22:0 Lyso PC and mRNA were loaded into the LNPs without drastically increasing the size or polydispersity index (PDI) of the LNPs (FIG. 1A-B). In all cases, LNPs prepared with mRNA were larger, and there was a slight increase in effective diameter when 22:0 Lyso PC was added to the LNPs (FIG. 1A). All LNPs were still nanoparticle sized, with all diameters <125 nm. Adding 22:0 Lyso PC did not impact the PDI of the LNPs (FIG. 1B), and all LNP formulations resulted in relatively uniform particle populations (PDI<0.3). In most cases, all size readings for the LNPs were relatively close by dynamic light scatter (DLS), with the exception of the LNP 20 formulation without mRNA. Therefore, in some embodiments, 22:0 Lyso PC is included in LNP formulations at a percent molarity above 20%, preferably above 25%, and more preferably at least 30%.

22:0 Lyso PC LNPs allow for hyperactivation of human moDCs. Human monocytes isolated from Leukopaks were differentiated into monocyte-derived DCs (moDCs) over 6 days in culture with GM-CSF and IL-4. Six days after differentiation, moDCs were collected and plated into 96-well flat-bottom plates at 1×10⁵ cells/well. moDCs were treated with or without 1 μg/mL R848, and with or without LNPs loaded with a hyperactivating lipid (or LNP vehicle control) in the presence or absence of eGFP mRNA. LNPs were dosed for a total amount of 50 μM or 100 μM 22:0 Lyso PC based on the loading content of the LNPs. Hyperactivation induced by LNPs was measured after 48 hrs in culture with the LNPs by assessing cytokine secretion from live moDCs.

Cell Viability was assessed by LDH present in the supernatants, and normalized to the wells treated with R848 alone (no LNPs). At the 50 μM treatment conditions, all wells had similar viability to the R848 alone treatment condition, regardless of the presence of LNPs or 22:0 Lyso PC in the LNPs (FIG. 2A). At the 100 μM dose, most treatments containing R848 were associated with a cell viability >75% (FIG. 2B). Though the viability of the LNP 40 condition fell below 75% when cultured without R848, when R848 was added to the wells the cell viability was above 75%. To measure the ability of the 22:0 Lyso PC delivered in LNPs to hyperactivate moDCs, IL-1β cytokine secretion was measured in the cell culture supernatants. Importantly, only live cells that were treated with R848 and LNPs containing 22:0 Lyso PC were able to produce IL-1β (FIG. 2C-2D). The IL-1β secretion was dose-dependent, with cells treated with 100 μM 22:0 Lyso PC able to produce more IL-1β than cells treated with 50 μM 22:0 Lyso PC. Importantly, IL-1β secretion was not impacted by the presence of mRNA in the LNPs, indicating that LNPs loaded with 22:0 Lyso PC and mRNA have increased immunogenicity compared to LNPs lacking 22:0 Lyso PC. In addition, increasing the molar ratio of 22:0 Lyso PC in the LNPs resulted in increased IL-1β secretion, indicating that even if the same total dose of 22:0 Lyso PC is delivered, that changing the amount of 22:0 Lyso PC in an LNP may change the immunogenicity of an LNP.

In addition to IL-1β, IL-6 secretion was measured in the cell culture supernatant after 48 hrs. Interestingly, adding 22:0 Lyso PC to an LNP increased the amount of IL-6 secreted in response to R848 (FIG. 2E-2F). moDCs treated LNPs without 22:0 Lyso PC produced IL-6 at a similar level to cells treated with R848 alone, indicating the LNP itself is not necessarily strongly immunogenic. However, when the LNPs were also loaded with 22:0 Lyso PC, the amount of IL-6 secreted increased significantly. In addition, this response is dose-dependent, with cells treated with 100 μM 22:0 Lyso PC able to produce more IL-6 than cells treated with 50 μM 22:0 Lyso PC.

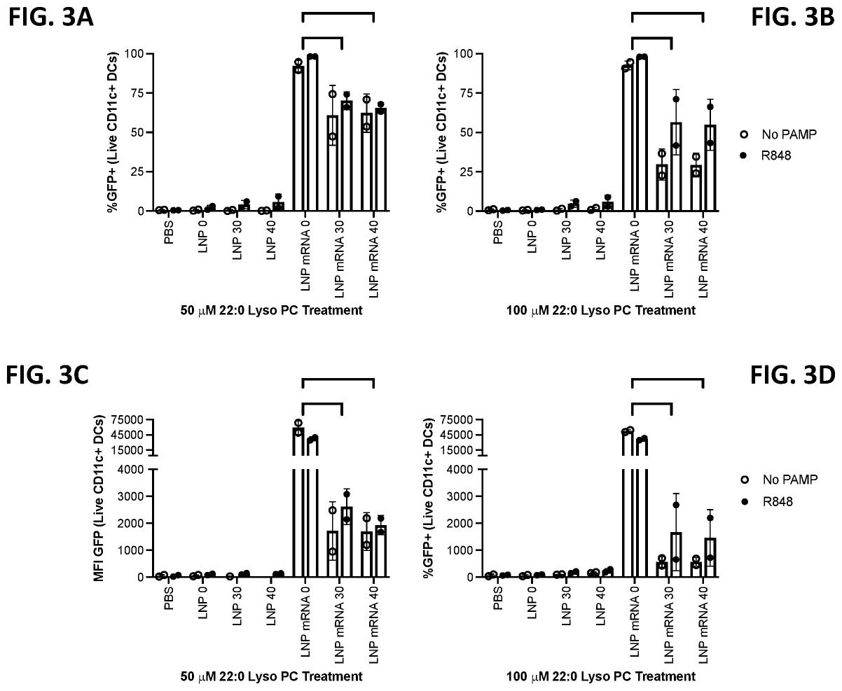

22:0 Lyso PC LNPs allow for mRNA translation and increase surface expression of activation markers in human moDCs. To determine if hyperactivation of moDCs would impact mRNA expression, levels of GFP were measured. Only cells that were treated with LNPs containing GFP mRNA resulted in GFP expression (FIG. 3A-3B). While treatment with 22:0 Lyso PC containing LNPs (LNP mRNA 30 and LNP mRNA 40) did decrease the percentage of cells positive for GFP, the majority of cells treated with mRNA LNPs were able to translate GFP mRNA into protein. Importantly, although the median fluorescence intensity (MFI) of GFP was lower for 22:0 Lyso PC containing LNPs (FIG. 3C-3D), the percentage of cells expressing mRNA remained high, indicating that the cells continue to express GFP, albeit at a lower rate. Given these moDCs are being hyperactivated at the same time that they are producing GFP, the lower level of GFP expression is not contemplated to translate into a markedly lower level of antigen (GFP) presentation at the cell surface. Interestingly, this was found to be dose-dependent, as increasing amounts of 22:0 Lyso PC resulted in lower levels of GFP expression, while increasing levels of IL-6 and IL-1β secretion. Importantly, moDCs treated with GFP mRNA-loaded LNPs are able to express GFP, regardless of 22:0 Lyso PC loading into LNPs.

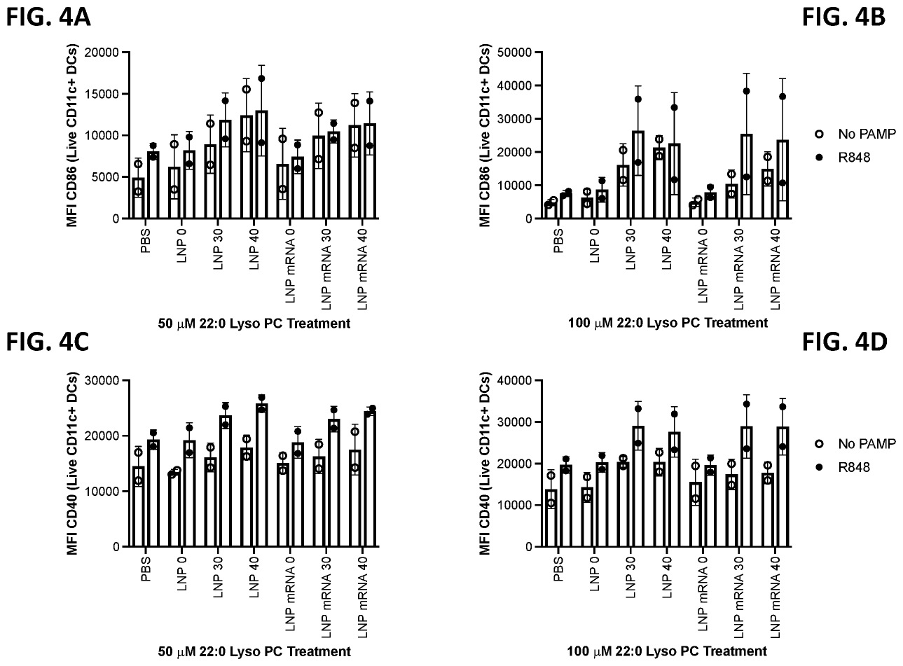

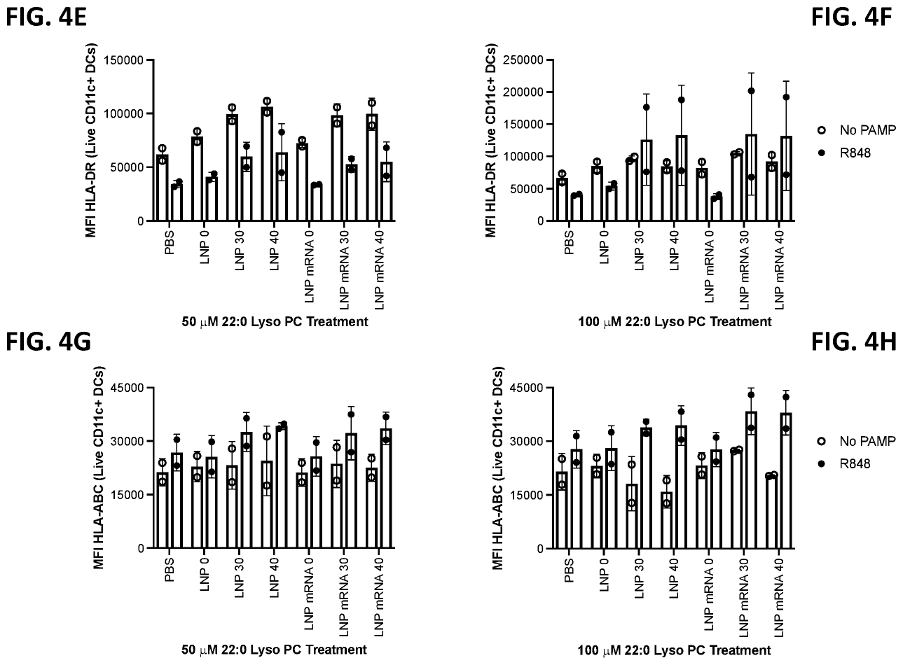

In parallel with the studies described above, moDCs were collected after 48 hrs in culture with LNPs, and live CD11c⁺ cells were assessed for surface expression of CD40, CD86, HLA-DR, and HLA-ABC. Levels of CD86 and CD40 on moDCs were measured to determine if hyperactivation would impact activation marker expression. With moDCs prepared from samples of two human donors, treatment with 22:0 Lyso PC loaded LNPs increased the median fluorescence intensity (MFI) of both CD86 (FIG. 4A-4B) and CD40 (FIG. 4C-4D), which is indicative of increases in co-stimulatory and activation marker expression following hyperactivation. In addition, the increase in CD86 and CD40 was dose dependent, as treatment with 100 μM 22:0 Lyso PC further increased CD86 and CD40 surface expression. Levels of HLA-DR and HLA-ABC on moDCs were measured to determine if hyperactivation would interfere with major histocompatibility complex (MHC) expression and therefore antigen presentation. Similar to activation marker expression, treatment of moDCs with 22:0 Lyso PC loaded LNPs increased the MFI of both HLA-DR (MHC-II) (FIG. 4E-4F) and HLA-ABC (MHC-I) (FIG. 4G-4H), which is indicative of an increase in expression of antigen presenting molecules following hyperactivation. The increase was also observed to be dose dependent, as treatment with 100 μM 22:0 Lyso PC further increased HLA-DR and HLA-ABC expression.

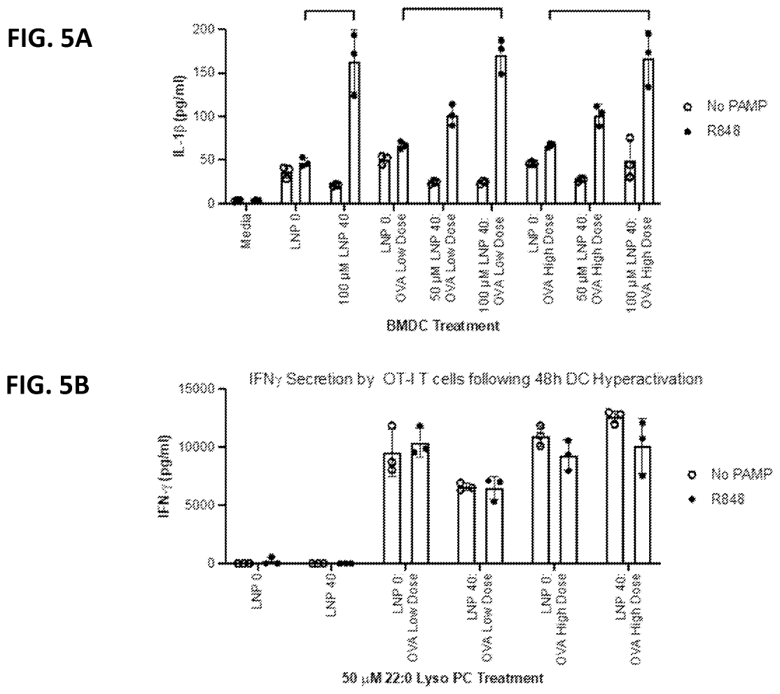

Murine DC:T cell co-cultures allow for antigen-specific T cell activation with 22:0 LPC loaded LNPs. To expand on observations made in in the human moDC system, studies were also conducted in a murine system. LNPs were loaded with 22:0 Lyso PC and two different concentrations of OVA mRNA (Table 2-2). Murine bone marrow-derived dendritic cells (BMDCs) were then hyperactivated with R848 and 22:0 Lyso PC at 50 μM or 100 μM delivered via LNPs containing a low or a high dose of OVA mRNA (Table 2-2). About 48 hrs post stimulation, supernatants were collected for measurement of IL-1β secretion. Importantly, only BMDCs that were treated with R848 and LNPs containing 22:0 Lyso PC were able to produce IL-1β at high levels (FIG. 5A). Cells that were treated with LNPs that did not contain 22:0 Lyso PC (LNP 0 formulations) did produce more IL-1β than cells treated with R848 alone. Addition of 22:0 Lyso PC to the LNPs resulted in significantly more IL-1β production. As was also seen in human moDCs, IL-1β secretion was dose-dependent, with cells treated with 100 μM 22:0 Lyso PC producing more IL-1β than cells treated with 50 μM. Additionally, IL-1β secretion was not impacted by the presence or dose of mRNA in the LNPs. This indicates that LNPs loaded with 22:0 Lyso PC and mRNA are more immunogenic than LNPs lacking 22:0 Lyso PC.

In addition, BMDCs that were hyperactivated for 48 hrs with LNPs containing 22:0 Lyso PC and different doses of OVA mRNA were co-cultured with OT-I CD8+ T cells collected from the spleens and lymph nodes of OT-I mice. All CD8+ T cells of OT-I mice are naïve (antigen-inexperienced) and specific for an OVA peptide. Hyperactivated DCs and CD8+ T cells were co-cultured for 72 hrs, after which the co-culture supernatants were collected. IFNγ secretion by CD8+ T cells in response to OVA presented by BMDCs was assessed. As expected, IFNγ secretion by CD8+ T cells was driven by the presence of OVA mRNA in the LNPs (FIG. 5B). Importantly, IFNγ secretion by activated CD8+ T cells was not impacted by the presence of 22:0 Lyso PC in LNPs. Interestingly, a 10-fold dose difference in mRNA between the low and high OVA mRNA doses did not impact IFNγ secretion. In combination, these results indicate that loading 22:0 Lyso PC into LNPs does not reduce antigen expression to a level where it would negatively impact antigen presentation at the DC cell surface, and thus permitting the activation of naïve, antigen-specific CD8+ T cells.

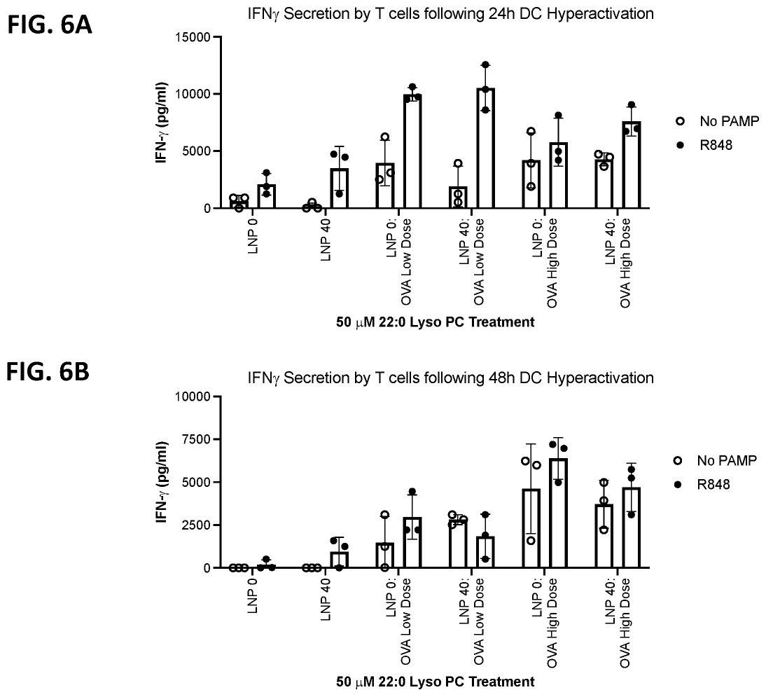

BMDCs that were hyperactivated for 48 hrs with LNPs containing 22:0 Lyso PC and different doses of OVA mRNA were also co-cultured with CD8+ T cells that were collected from the spleens and lymph nodes of mice previously immunized with ovalbumin in IFA. The OVA-IFA immunization scheme generates a population of antigen-experienced OVA-specific CD8+ T cells at physiologically relevant levels. Hyperactivated BMDCs and CD8+ T cells were co-cultured for 96 hrs, after which the co-culture supernatants were collected. IFNγ secretion by CD8+ T cells that were reactivated in response to OVA presented by BMDCs was assessed. Importantly, IFNγ secretion by reactivated CD8+ T cells was largely driven by the dose of OVA mRNA delivered, and was not impacted by the presence of 22:0 Lyso PC in LNPs (FIG. 6A-6B). Again, this data indicates that loading 22:0 Lyso PC into LNPs does not interfere with antigen expression and presentation from mRNA encoding the antigen.

Taken together, these results indicate that use of a more immunogenic LNP, loaded with a LPC with a single acyl chain (e.g., 22:0 Lyso PC) in addition to mRNA encoding an antigen have benefits over LNPs that do not contain a hyperactivating lipid. The impacts of hyperactivating LNP are expected to be more apparent in vivo. In particular, increased migration to lymph nodes and IL-1β secretion by DCs that have taken up the hyperactivating LNPs is expected to more potently guide de novo antigen-specific memory T cell generation and antigen-specific T cell reactivation.

We claim:

1. A composition comprising an mRNA encapsulated in a lipid nanoparticle (LNP), wherein the mRNA comprises a coding region of an antigen, and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

2. A composition comprising an mRNA and a TLR7/8 agonist encapsulated in a lipid nanoparticle (LNP), wherein the mRNA comprises a coding region of an antigen, and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

3. A composition comprising an mRNA encapsulated in a lipid nanoparticle (LNP), and a TLR7/8 agonist, wherein the mRNA comprises a coding region of an antigen, and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

4. A composition comprising a TLR7/8 agonist encapsulated in a lipid nanoparticle (LNP), and the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

5. A composition comprising a lipid nanoparticle (LNP) and a TLR7/8 agonist, wherein the LNP comprises a first phospholipid, and at least one lipid selected from the group consisting of an ionizable lipid, a second phospholipid, a pegylated lipid, a structural lipid, and mixtures thereof, wherein the first phospholipid comprises a lysophosphatidylcholine (LPC) with a single acyl chain, and the acyl chain is a C13-C24 acyl chain.

6. The composition of any one of claims 1-5, wherein the at least one lipid comprises an ionizable lipid, a second phospholipid, a pegylated lipid, and a structural lipid.

7. The composition of any one of claims 1-6, wherein the ionizable lipid comprises:
i) 8-[(2-hydroxyethyl)[6-oxo-6-(undecyloxy)hexyl]amino]-octanoic acid, 1-octylnonyl ester (SM-102) or analogs or derivatives thereof; and/or
ii) 6-((2-hexyldecanoyl)oxy)-N-(6-((2-hexyldecanoyl)oxy)hexyl)-N-(4-hydroxybutyl)hexan-1-aminium (ALC-0315) or analogs or derivatives thereof.

8. The composition of any one of claims 1-7, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.

9. The composition of any one of claims 1-7, wherein the pegylated lipid comprises polyethylene glycol [PEG] 2000 dimyristoyl glycerol [DMG].

10. The composition of any one of claims 1-9, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.

11. The composition of any one of claims 1-9, wherein the structural lipid comprises cholesterol.

12. The composition of any one of claims 1-11, wherein the second phospholipid comprises:
i) a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin; and
ii) one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

13. The composition of any one of claims 1-11, wherein the second phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
combinations thereof.

14. The composition of any one of claims 1-13, wherein the second phospholipid comprises 1,2-distearoyl-sn-glycero-3-phosphocholine (DSPC).

15. The composition of any one of claims 1-14, wherein the composition further comprises at least one excipient.

16. The composition of claim 15, wherein the excipient comprises sucrose.

17. A composition comprising:
i) an mRNA complexed with one or more lipids (RNA-Lipoplex); and
ii) a lysophosphatidylcholine (LPC) with a single C13-C24 acyl chain,
wherein the mRNA comprises a coding region of an antigen, and the one or more lipids comprise a first lipid and a second lipid.

18. A composition comprising:
i) an mRNA complexed with one or more lipids (RNA-Lipoplex);
ii) a lysophosphatidylcholine (LPC) with a single C13-C24 acyl chain; and
iii) a TLR7/8 agonist,
wherein the mRNA comprises a coding region of an antigen, and the one or more lipids comprise a first lipid and a second lipid.

19. The composition of claim 17 or claim 18, wherein the first lipid is a cationic lipid, and the second lipid is a neutral or anionic lipid.

20. The composition of claim 19, wherein the cationic lipid comprises one or both of:
i) 1,2-di-O-octadecenyl-3-trimethylammonium propane (DOTMA) or analogs or derivatives thereof; and
ii) 1,2-dioleoyl-3-trimethylammonium propane (DOTAP) or analogs or derivatives thereof.

21. The composition of claim 19 or claim 20, wherein the neutral or anionic lipid comprises:
i) 1,2-di-(9Z-octadecenoyl)-sn-glycero-3-phosphoethanolamine (DOPE) or analogs or derivatives thereof; and/or
ii) cholesterol or analogs or derivatives thereof; and/or
iii) 1,2-dioleoyl-sn-glycero-3-phosphocholine (DOPC) or analogs or derivatives thereof.

22. The composition of any one of claims 1-21, wherein the acyl chain of the LPC is a C21-C24 acyl chain.

23. The composition of any one of claims 1-21, wherein the acyl chain of the LPC is a C22 acyl chain.

24. The composition of any one of claims 1-23, wherein the acyl chain of the LPC is fully saturated.

25. The composition of claim 24, wherein the LPC comprises 1-behenoyl-2-hydroxy-sn-glycero-3-phosphocholine [LPC(22:0)].

26. The composition of any one of claims 1-25, wherein the TLR7/8 agonist is a small molecule with a molecule weight of 900 daltons or less

27. The composition of any one of claims 1-25, wherein the TLR7/8 agonist comprises an imidazoquinoline compound.

28. The composition of claim 27, wherein the TLR7/8 agonist comprises resiquimod (R848).

29. The composition of any one of claims 1-28, wherein the LPC comprises LPC(22:0), and the TLR7/8 agonist comprises resiquimod (R848).

30. The composition of any one of claims 1-28, wherein the antigen is a tumor antigen.

31. The composition of any one of claims 1-28, wherein the tumor antigen is a neoantigen.

32. The composition of any one of claims 1-28, wherein the antigen comprises a microbial antigen.

33. The composition of claim 32, wherein the microbial antigen comprises a viral antigen, a bacterial antigen, a protozoan antigen, or a fungal antigen.

34. The composition of claim 32, wherein the microbial antigen comprises a surface antigen.

35. The composition of any one of claims 1-34, wherein the mRNA comprises a 5′ untranslated region (5′UTR) at the 5′ end of the coding region and a 3′ untranslated region (3′UTR) at the 3′ end of the coding region.

36. The composition of any one of claims 1-35, wherein the mRNA comprises a 5′ cap structure.

37. The composition of any one of claims 1-36, wherein the mRNA comprises a polyA tail.

38. The composition of any one of claims 1-37, wherein the composition does not comprise lipopolysaccharide (LPS) or monophosphoryl lipid A (MPLA).

39. The composition of any one of claims 1-38, wherein the composition does not comprise oxidized 1-palmitoyl-2-arachidonoyl-sn-glycero-3-phosphorylcholine (oxPAPC) or a species of oxPAPC.

40. The composition of claim 39, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl]2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl]2-(trimethylazaniumyl)ethyl phosphate (POVPC), [(2R)-2-(4-carboxybutanoyloxy)-3-hexadecanoyloxy propyl]2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl]2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl]2-(trimethylazaniumyl)ethyl phosphate (PEIPC) and/or 1-palmitoyl-2-azelaoyl-sn-glycero-3-phosphocholine (PAzePC).

41. The composition of any one of claims 1-40, wherein the composition does not comprise an antigen.

42. A pharmaceutical formulation comprising the composition of any one of claims 1-41, and a pharmaceutically acceptable excipient.

43. A method for production of hyperactivated dendritic cells, the method comprising contacting the dendritic cells with an effective amount of the composition of any of the preceding claims to produce hyperactivated dendritic cells, wherein the hyperactivated dendritic cells secrete IL-1beta without undergoing cell death within about 48 hours of exposure.

44. The method of claim 43, wherein the dendritic cells are contacted in vivo with the composition.

45. The method of claim 43, wherein the dendritic cells are contacted ex vivo with the composition.

46. A pharmaceutical formulation comprising at least 10\hat{ }3, 10\hat{ }4, 10\hat{ }5 or 10\hat{ }6 of the hyperactivated dendritic cells produced by the method of claim 45, and a pharmaceutically acceptable excipient.

47. A method of stimulating an immune response against an antigen, comprising administering an effective amount of the pharmaceutical formulation of claim 42 or claim 46 to an individual in need thereof to stimulate the immune response against the antigen.

48. A method of treating cancer, comprising administering an effective amount of the pharmaceutical formulation of claim 42 or claim 46 to an individual in need thereof to treat the cancer.

49. A method of inhibiting abnormal cell proliferation, comprising administering an effective amount of the pharmaceutical formulation of claim 42 or claim 46 to an individual in need thereof to inhibit abnormal cell proliferation.

50. A method of treating or preventing an infectious disease, comprising administering an effective amount of the pharmaceutical formulation of claim 42 to an individual in need thereof to treat or prevent the infectious disease.

51. The method of claim 50, wherein the infectious disease is a viral disease.

52. The method of claim 51, wherein the infectious disease is a bacterial disease.

53. The method or pharmaceutical formulation of any one of claims 43-49, wherein the dendritic cells are mammalian cells.

54. The method or pharmaceutical formulation of claim 53, wherein the mammalian cells are human cells.

55. The method of any one of claims 47-53, wherein the individual is mammal.

56. The method of claim 55, wherein the mammal is a human.

57. The method of claim 55, wherein the mammal is a dog or a cat.

58. The composition, formulation, or method or use of any one of claims 1-57, wherein the composition does not comprise a protein.

59. The composition, formulation, method or use of any one of claims 1-58, wherein the LNP has an effective diameter of less than about 500 nanometers, optionally from about 5 to about 500 nanometers, optionally from about 10 to about 400 nanometers, optionally from about 20 to about 300 nanometers, or optionally from about 25 to about 250 nanometers.

60. The composition, formulation, method or use of claim 59, wherein the LNP has an effective diameter of less than about 250 nanometers.

61. The composition, formulation, method or use of claim 60, wherein the LNP has an effective diameter of less than about 125 nanometers.

62. The composition, formulation, method or use of claim 61, wherein the LNP has an effective diameter of from about 20 to about 120 nanometers.
